(19) United States
(12) Patent Application Publication
(10) Pub. No.: US 2008/0085867 A1
(43) Pub. Date: Apr. 10, 2008

(54) Early detection and prognosis of colon cancers

(75) Inventors: Stephen B. Baylin, Baltimore, MD (US); Wim van Criekinge, Sart-Tilman (Liege) (BE); Kornel E. Schuebel, Baltimore, MD (US); Leslie Cope, Baltimore, MD (US); Hiromu Suzuki, Sapporo (JP); James G. Herman, Baltimore, MD (US)

Correspondence Address: BANNER & WITCOFF, LTD., 1100 13th STREET, N.W., SUITE 1200, WASHINGTON, DC, 20005-4051, US

(73) Assignees: The Johns Hopkins University, Baltimore, MD (US); Onomethylome Sciences, S.A., Leuven (BE)

(21) Appl. No.: 11/826,508

(22) Filed: Jul. 16, 2007

Related U.S. Application Data
(60) Provisional application No. 60/807,376, filed on Jul. 14, 2006.

(51) Int. Cl. A61K 31/7052; A61K 31/195; A61K 31/7068; C12Q 1/68; C12N 5/00; A61K 31/216

(52) U.S. Cl. 514/44; 435/325; 435/6; 514/49; 514/535; 514/562

(57) ABSTRACT

A genome wide microarray gene expression approach for human colorectal cancer cells was used to identify hundreds of hypermethylated genes for colon cancer. We compared isogenic cells altered pharmacologically versus genetically to induce genomic demethylation, to pinpoint genes activated by DNA demethylation, but not by inhibition of class I and II histone deacetylases (HDACs). We achieve an 82% success rate in predicting genes with densely hypermethylated CpG islands and complete gene silencing. The genes are similarly hypermethylated in primary tumors and have previously undetected tumor suppressor functions. The genes can be used diagnostically to detect cancer, pre-cancer, and likelihood of developing cancer.

This application claims the benefit of U.S. provisional application Ser. No. 60/807,376 filed Jul. 14, 2006.

This invention was made using U.S. government funds under grant ESI 1858 from the National Institute of Environmental Health Sciences under grant CA043318 from the National Cancer Institute. The U.S. government retains certain rights to the invention under the terms of these grants.

### TECHNICAL FIELD OF THE INVENTION

This invention is related to the area of cancer diagnostics and therapeutics. In particular, it relates to aberrant methylation patterns of particular genes in colon cancer and pre-cancer.

### BACKGROUND OF THE INVENTION

DNA Methylation and its Role in Carcinogenesis

The information to make the cells of all living organisms is contained in their DNA. DNA is made up of a unique sequence of four bases: adenine (A), guanine (G), thymine (T) and cytosine (C). These bases are paired A to T and G to C on the two strands that form the DNA double helix. Strands of these pairs store information to make specific molecules grouped into regions called genes. Within each cell, there are processes that control what gene is turned on, or expressed, thus defining the unique function of the cell. One of these control mechanisms is provided by adding a methyl group onto cytosine (C). The methyl group tagged C can be written as mC.

DNA methylation plays an important role in determining whether some genes are expressed or not. By turning genes off that are not needed, DNA methylation is an essential control mechanism for the normal development and functioning of organisms. Alternatively, abnormal DNA methylation is one of the mechanisms underlying the changes observed with aging and development of many cancers.

Cancers have historically been linked to genetic changes caused by chromosomal mutations within the DNA. Mutations, hereditary or acquired, can lead to the loss of expression of genes critical for maintaining a healthy state. Evidence now supports that a relatively large number of cancers are caused by inappropriate DNA methylation, frequently near DNA mutations. In many cases, hyper-methylation of DNA incorrectly switches off critical genes, such as tumor suppressor genes or DNA repair genes, allowing cancers to develop and progress. This non-mutational process for controlling gene expression is described as epigenetics.

DNA methylation is a chemical modification of DNA performed by enzymes called methyltransferases, in which a methyl group (m) is added to certain cytosines (C) of DNA. This non-mutational (epigenetic) process (mC) is a critical factor in gene expression regulation. See, J. G. Herman, Seminars in Cancer Biology, 9: 359-67, 1999.

Although the phenomenon of gene methylation has attracted the attention of cancer researchers for some time, its true role in the progression of human cancers is just now being recognized. In normal cells, methylation occurs predominantly in regions of DNA that have few CG base repeats, while CpG islands, regions of DNA that have long repeats of CG bases, remain non-methylated. Gene promoter regions that control protein expression are often CpG island-rich. Aberrant methylation of these normally non-methylated CpG islands in the promoter region causes transcriptional inactivation or silencing of certain tumor suppressor expression in human cancers.

Genes that are hypermethylated in tumor cells are strongly specific to the tissue of origin of the tumor. Molecular signatures of cancers of all types can be used to improve cancer detection, the assessment of cancer risk and response to therapy. Promoter hypermethylation events provide some of the most promising markers for such purposes.

Promoter Gene Hypermethylation: Promising Tumor Markers

Information regarding the hypermethylation of specific promoter genes can be beneficial to diagnosis, prognosis, and treatment of various cancers. Methylation of specific gene promoter regions can occur early and often in carcinogenesis making these markers ideal targets for cancer diagnostics.

Methylation patterns are tumor specific. Positive signals are always found in the same location of a gene. Real time PCR-based methods are highly sensitive, quantitative, and suitable for clinical use. DNA is stable and is found intact in readily available fluids (e.g., serum, sputum, stool, blood, and urine) and paraffin embedded tissues. Panels of pertinent gene markers may cover most human cancers.

Diagnosis

Key to improving the clinical outcome in patients with cancer is diagnosis at its earliest stage, while it is still localized and readily treatable. The characteristics noted above provide the means for a more accurate screening and surveillance program by identifying higher-risk patients on a molecular basis. It could also provide justification for more definitive follow up of patients who have molecular but not yet all the pathological or clinical features associated with malignancy.

At present, early detection of colorectal cancer is carried out by (1) the “fecal occult blood test” (FOBT), which has a very low sensitivity and specificity, (2) by sigmoidoscopy and/or colonoscopy which is invasive and expensive (and limited in supply), (3) by X-ray detection after double-contrast barium enema, which allows only for the detection of rather large polyps, or CT-colonography (also called virtual colonoscopy) which is still experimental, and (4) by a gene mutation analysis test called PreGen-Plus (Exact Sciences; LabCorp) which is costly and of limited sensitivity.

Predicting Treatment Response

Information about how a cancer develops through molecular events could allow a clinician to predict more accurately how such a cancer is likely to respond to specific chemotherapeutic agents. In this way, a regimen based on knowledge of the tumor's chemosensitivity could be rationally designed. Studies have shown that hypermethylation of the MGMT promoter in glioma patients is indicative of a good response to therapy, greater overall survival and a longer time to progression.

There is a continuing need in the art for new diagnostic and prognostic markers and therapeutic targets for cancer to improve management of patient care.

### SUMMARY OF THE INVENTION

According to one embodiment of the invention, a method is provided for identifying colorectal cancer or its precursor, or predisposition to colorectal cancer. Epigenetic silencing of at least one gene listed in Table 1 is detected in a test sample. The test sample contains colorectal cells or nucleic acids from colorectal cells. The cells or nucleic acids in the test sample are identified as neoplastic, precursor to neoplastic, or predisposed to neoplasia.

Another embodiment of the invention is a method of reducing or inhibiting neoplastic growth of a cell which exhibits epigenetic silenced transcription of at least one gene associated with a cancer. An epigenetically silenced gene is determined in a cell. The epigenetically silenced gene is selected from the group consisting of those listed in Table 1. Expression of a polypeptide encoded by the epigenetic silenced gene is restored in the cell by contacting the cell with a CpG dinucleotide demethylating agent, thereby reducing or inhibiting unregulated growth of the cell.

Another embodiment of the invention is a method of reducing or inhibiting neoplastic growth of a cell which exhibits epigenetic silenced transcription of at least one gene associated with a cancer. An epigenetically silenced gene is determined in a cell. The gene is selected from the group consisting of those listed in Table 1. A polynucleotide encoding a polypeptide is introduced into the cell. The polypeptide is encoded by said gene. The polypeptide is expressed in the cell thereby restoring expression of the polypeptide in the cell.

According to yet another aspect of the invention a method of treating a cancer patient is provided. A cancer cell in the patient is determined to have an epigenetic silenced gene selected from the group consisting of those listed in Table 1. A demethylating agent is administered to the patient in sufficient amounts to restore expression of the epigenetic silenced gene in the patient's cancer cells.

Still another embodiment of the invention is another method of treating a cancer patient. A cancer cell in the patient is determined to have an epigenetic silenced gene selected from the group consisting of those listed in Table 1. A polynucleotide encoding a polypeptide is administered to the patient. The polypeptide is encoded by the epigenetic silenced gene. The polypeptide is expressed in the patient's tumor, thereby restoring expression of the polypeptide in the cancer.

The invention also provide a method for selecting a therapeutic strategy for treating a cancer patient. A gene whose expression in cancer cells of the patient is reactivated by a demethylating agent is identified. The gene is selected from the group consisting of those listed in Table 1. A therapeutic agent which increases expression of the gene is selected for treating said cancer patient.

The present invention also provides a kit for assessing methylation in a cell sample. The kit provides in a package: (1) a reagent that (a) modifies methylated cytosine residues but not non-methylated cytosine residues, or that (b); modifies non-methylated cytosine residues but not methylated cytosine residues; and (2) a pair of oligonucleotide primers that specifically hybridizes under amplification conditions to a region of a gene selected from the group consisting of those listed in Table 1. The region of the gene is within about 1 kb of said gene's transcription start site.

These and other embodiments which will be apparent to those of skill in the art upon reading the specification provide the art with tools and methods for detection, diagnosis, prognosis, therapy, and drug selection pertaining to neoplastic cells and cancers.

### BRIEF DESCRIPTION OF THE FIGURES AND TABLE

FIG. 1A-1D Approach for identification of the human cancer cell hypermethylome. FIG. 1A, RNA from the indicated cell lines were processed and hybridized with Agilent 44K human microarray chips as shown. Parental HCT116 cells are indicated as wild type (WT) and the genotype for DNA methyltransferase 1 or 3b deficient cells is indicated. DKO cells are doubly deficient for DNMT1 and DNMT3b. HCT116 cells were treated with trichostatin A (TSA) or 5-azadeoxycytidine (DAC) and hybridized against mock-treated cells. FIG. 1B, Gene expression peak in demethylated HCT116 cells. Scatter plot indicating the location of all gene expression changes (black), average (red dots) or individual (blue) of DKO samples with greater than 4 fold expression change plotted in three dimensions. FIG. 1C, Pharmacological treatment reveals the cancer cell hypermethylome. Gene expression changes from HCT116 cells treated with TSA or AZA were plotted and overlaid with various data sets. Yellow spots indicate genes from DKO cells with 2 fold changes and above. Green spots indicate experimentally verified genes derived from the hypermethylome, while red spots indicate those that did not verify. Blue spots indicate the location of the 11 guide genes used in this study. FIG. 1D, Relationship of different datasets used in this study. Relatedness of whole transcriptome expression patterns verified by dendrogram analysis. DNA methyltransferase single knockout, DKO and AZA treatment, and TSA treatment induced three distinct categories of gene expression changes.

FIG. 2A-2E. Genes that guide and verify the identity of the hypermethylome. Hypermethylated guide genes identified in HCT116 cells used in this study are indicated in FIG. 2A, Gene names, Agilent ID numbers, GENBANK accession numbers, and references are indicated. Location of the guide genes is indicated in blue plotted against gene expression changes in AZA treated (FIG. 2B) or DKO cells (FIG. 2C). Green circles indicate the location of the four guide genes with DAC induced expression increases in the higher tier of the no TSA response zone. FIG. 2D, Relative position of the guide genes plotted by fold change in demethylated (DKO or AZA-treated) cells. The green circle indicates the location of the four informative guide genes. FIG. 2E List of candidate hypermethylome genes used for verification of expression and methylation. Agilent ID, gene name and description are indicated on the left panel. Gene expression was verified by RT-PCR and methylation by MSP. Water and in vitro methylated DNA (IVD) were used as controls. Green arrows identify genes that did verify the array results, red arrows those that did not.

FIG. 3A-3E. Epigenetic inactivation of Neuralized and FOXL2 genes in colorectal cancer cell lines and tumors. FIG. 3A and FIG. 3C, Cell line abbreviations are indicated at the top, with the upper panel indicating methylation tested by MSP and expression tested by RT-PCR before (+) and after (−) DAC treatment. DKO and water (H2O) controls are indicated on the right panel. Graphical display of the Neuralized (FIG. 3B) or FOXL2 CpG island (FIG. 3D), with bisulfite sequencing primers indicated in black, MSP primers indicated in red and CpG nucleotides as open circles. Transcription start sites are indicated with a green square, and the 5′ and 3′ ends are indicated by numbers with respect to the transcription start site. Bisulfite sequencing results in cell lines (HCT116, RKO or DKO) or human tissues (colon or rectum); unmethylated CpGs are indicated by open circles, methylated CpGs by shaded circles. FIG. 3E Methylation of Neuralized and FOXL2 in human colorectal tumor samples. Tumors were classified as being microsatellite stable (MSS) or having microsatellite instability (MSI) according to Bat26 microsatellite expansion and MLH1 protein staining.

FIG. 4A-4D Tumor suppressor activity of FOXL2 and Neuralized gene products. FIG. 4A, Expression vectors encoding full length Neuralized or FOXL2, or empty vector were transfected into HCT116 cells, selected for Hygromycin resistance and stained. FIG. 4B, Resulting colonies were visualized by light microscopy. FIG. 4C, Colony number resulting from transfection with the indicated plasmid in HCT116 cells, or FIG. 4D RKO or DLD1 cells.

FIG. 5 (Table 1.) Methylation markers for early detection and prognosis of colon cancer or pre-cancer or the risk of cancer.

### DETAILED DESCRIPTION OF THE INVENTION

The inventors have discovered a set of genes whose transcription is epigenetically silenced in cancers, cancer precursors, and pre-cancers. All of the identified genes are shown in Table 1. Detection of epigenetic silencing of at least 1, 2, 3, 4, 5, 6, 7, 8, 9, or 10 of such genes can be used as an indication of cancer or pre-cancer or risk of developing cancer. Among the genes identified in Table 1, A_{—}23_P132956 UCHL1 Homo sapiens ubiquitin carboxyl-terminal esteraseL1 (ubiquitin thiolesterase); A_{—}23_P29046 CBR1 Homo sapiens carbonyl reductase; A_{—}23_P92499 TLR2 Homo sapiens toll-like receptor 2; A_{—}23_P393620 TFPI2 Homo sapiens tissue factor pathway inhibitor 2; A_{—}23_P120243 HOXD1 Homo sapiens homeo box D1; A_{—}23_P115407 GSTM3 Homo sapiens glutathione S-transferase M3; A_{—}23_P153320 ICAM1 Homo sapiens intercellular adhesion molecule 1 (CD54), human rhinovirus receptor; A_{—}23_P143981 FBLN2 Homo sapiens fibulin 2; A_{—}23_P110052 FOXL2 Homo sapiens forkhead box L2; A_{—}23_P138492 NEURL Neuralized homolog; A_{—}32_P184916 GNB4 Homo sapiens guanine nucleotide binding protein (G protein), beta polypeptide 4; and A_{—}24_P938403 JPH3 Homo sapiens junctophilin 3 provide high specificity.

Epigenetic silencing of a gene can be determined by any method known in the art. One method is to determine that a gene which is expressed in normal cells or other control cells is less expressed or not expressed in tumor cells. This method does not, on its own, however, indicate that the silencing is epigenetic, as the mechanism of the silencing could be genetic, for example, by somatic mutation. One method to determine that the silencing is epigenetic is to treat with a reagent, such as DAC (5′-deazacytidine), or with a reagent which changes the histone acetylation status of cellular DNA or any other treatment affecting epigenetic mechanisms present in cells, and observe that the silencing is reversed, i.e., that the expression of the gene is reactivated or restored. Another means to determine epigenetic silencing is to determine the presence of methylated CpG dinucleotide motifs in the silenced gene. Typically these reside near the transcription start site, for example, within about 1 kbp, within about 750 bp, or within about 500 bp. Once a gene has been identified as the target of epigenetic silencing in tumor cells, determination of reduced expression can be used as an indicator of epigenetic silencing.

Expression of a gene can be assessed using any means known in the art. Typically expression is assessed and compared in test samples and control samples which may be normal, non-malignant cells. Either mRNA or protein can be measured. Methods employing hybridization to nucleic acid probes can be employed for measuring specific mRNAs. Such methods include using nucleic acid probe arrays (microarray technology), in situ hybridization, and using Northern blots. Messenger RNA can also be assessed using amplification techniques, such as RT-PCR. Advances in genomic technologies now permit the simultaneous analysis of thousands of genes, although many are based on the same concept of specific probe-target hybridization. Sequencing-based methods are an alternative; these methods started with the use of expressed sequence tags (ESTs), and now include methods based on short tags, such as serial analysis of gene expression (SAGE) and massively parallel signature sequencing (MPSS). Differential display techniques provide yet another means of analyzing gene expression; this family of techniques is based on random amplification of cDNA fragments generated by restriction digestion, and bands that differ between two tissues identify cDNAs of interest. Specific proteins can be assessed using any convenient method including immunoassays and immuno-cytochemistry but are not limited to that. Most such methods will employ antibodies which are specific for the particular protein or protein fragments. The sequences of the mRNA (cDNA) and proteins of the markers of the present invention are known in the art and publicly available.

Methylation-sensitive restriction endonucleases can be used to detect methylated CpG dinucleotide motifs. Such endonucleases may either preferentially cleave methylated recognition sites relative to non-methylated recognition sites or preferentially cleave non-methylated relative to methylated recognition sites. Examples of the former are Acc III, Ban I, BstN I, Msp I, and Xma I. Examples of the latter are Acc II, Ava I, BssH II, BstU I, Hpa I, and Not I. Alternatively, chemical reagents can be used which selectively modify either the methylated or non-methylated form of CpG dinucleotide motifs.

Modified products can be detected directly, or after a further reaction which creates products which are easily distinguishable. Means which detect altered size and/or charge can be used to detect modified products, including but not limited to electrophoresis, chromatography, and mass spectrometry. Other means which are reliant on specific sequences can be used, including but not limited to hybridization, amplification, sequencing, and ligase chain reaction, Combinations of such techniques can be uses as is desired. Examples of such chemical reagents for selective modification include hydrazine and bisulfite ions. Hydrazine-modified DNA can be treated with piperidine to cleave it. Bisulfite ion-treated DNA can be treated with alkali.

Other techniques which can be used include technologies suitable for detecting DNA methylation with the use of bisulfite treatment include MSP, Mass Array, MethylLight, QAMA (quantitative analysis of methylated alleles), ERMA (enzymatic regional methylation assay), HeavyMethyl, pyrosequencing technology, MS-SNuPE, Methylquant, oligonucleotide-based microarray.

Methylation-specific PCR (MSP) is a bisulfite conversion-based PCR technique for the analysis of DNA methylation. After bisulfite treatment of DNA, an unmethylated cytosine will be converted to uracil and a methylated cytosine will be unaffected. For a MSP, two primer pairs are required: one pair with a primer complementary to methylated DNA, which contains cytosine residues, and the second pair with a primer complementary to unmethylated DNA, where cytosine residues have been converted to uracil. One performs two separate PCR reactions using each primer pair. Successful PCR amplification using the primer pair complementary to the DNA containing cytosine indicates methylation. Successful PCR amplification from the primer pair complementary to the DNA containing uracil indicates no methylation.

Methylation-Sensitive Single Nucleotide Primer Extension (Ms-SNuPE) is based on bisulfite treatment of DNA, a PCR reaction, and single nucleotide primer extension. After bisulfite treatment of DNA, which converts an unmethylated cytosine to uracil, while methylated cytosine residues remain unaffected, a PCR reaction is performed using primers to amplify a region that is potentially methylated. The resulting PCR product is used as a template for single nucleotide primer extension using a primer positioned directly 5′ of a potential methylation site. Single nucleotide primer extension proceeds with either [32P]dCTP or [32P]dTTP and is subsequently analyzed via electrophoresis and radiography. Primer extension incorporating dCTP indicates that a methylated cytosine is present in the template DNA, while incorporation of dTTP indicates the presence of an unmethylated cytosine that was converted to uracil. Gonzalgo, M and Jones, P. Rapid quantitation of methylation differences at specific sites using methylation-sensitive single nucleotide primer extension (Ms-SNuPE). See Gonzalgo and Jones, Rapid quantitation of methylation differences at specific sites using methylation-sensitive single nuclear primer extension. 1997 Nuc Acid Res, 25, 12.

The MassARRAY technique is based on bisulfite treatment of genomic DNA followed by PCR amplification. One PCR primer contains a T7 promoter sequence so a resulting PCR product will contain a T7 promoter. The PCR product is then used as a template for in vitro RNA transcription. RNaseA is used to cleave the in vitro transcribed RNA in a base specific fashion, generating specific RNA cleavage products. The RNA cleavage products are analyzed via MALDI-TOF mass spectrometry. RNA transcribed from the template DNA will have a different nucleotide composition depending on whether the genomic DNA template was methylated or non-methylated (cytosine or uracil, respectively) and this results in a different mass spectrometry signal pattern. See Ehrich, M. et al. 2005. Introduction to DNA methylation analysis using the MassARRAY system. SEQUENOM™ product preview note.

The methylation-specific oligonucleotide microarray technique begins with bisulfite-treatment of genomic DNA. The DNA is then used as a template for a PCR reaction. After bisulfite treatment, an unmethylated cytosine is converted to uracil and a methylated cytosine will remain the same because it is not converted by the bisulfite treatment. The PCR product is hybridized to a set of oligonucleotide probes that discriminate between the thymine, which is from unmethylated DNA, and the bisulfite-resistant cytosine, which is from methylated DNA, at specific nucleotide positions. Quantitative differences in hybridization are determined by fluorescence analysis. See Gitan R S et al. Methylation-specific oligonucleotide microarray: a new potential for high-throughput methylation analysis. 2006 Genome Research 12:158-164.

MethyLight is a fluorescence-based real-time PCR technique that is capable of quantitating DNA methylation at a particular locus. Genomic DNA is treated with sodium bisulfite, which converts an unmethylated cytosine to uracil, while methylated cytosine residues remain unaffected. The oligonucleotides are designed to be complementary to the DNA in a methylation-specific manner: one oligonucleotide is complementary to sequence containing uracil and another oligonucleotide is complementary to sequence containing cytosine. Generation of a PCR product is dependent on the methylation status of the template DNA. Fluorogenic PCR primers can be utilized, or a fluorogenic oligonucleotide probe, which is interpositioned between two PCR primers, can be utilized for a fluorescent readout. See Trinh B. et al. DNA methylation analysis by MethyLight technology. Methods. 2001 December; 25(4):

Quantitative Analysis of Methylated Alleles (QAMA) is an improvement on MethyLight technology. QAMA relies on interpositioned probes that are designed with minor groove binder technology. Minor groove binder technology is based on naturally occurring antibiotics that preferentially bind to the minor groove of double stranded DNA. These antibiotics are attached to either the 5′ or 3′ terminus of DNA probes, stabilizing the DNA duplex formed by these probes hybridizing to their complementary targets, allowing the use of shorter probes with higher sensitivity to mismatches. After bisulfite-treatment of genomic DNA, this type of interpositioned probe can be used in a MethyLight real-time PCR reaction to discriminate the methylation status of single CpG dinucleotides. See Zeschnigk M. et al. A novel real-time PCR assay for quantitative analysis of methylated alleles (QAMA): analysis of the retinoblastoma locus. 2004. Nuc Acid Res 32, 16.

HeavyMethyl technology is a variation on the methylation-specific PCR which relies on non-extendable oligonucleotides to provide methylation detection. DNA is first treated with sodium bisulfite, which converts an unmethylated cytosine to uracil, while methylated cytosine residues remain unaffected. The non-extendable oligonucleotides are designed to be complementary to the DNA in a methylation-specific manner: one non-extendable oligonucleotide is complementary to sequence containing uracil and another non-extendable oligonucleotide is complementary to sequence containing cytosine. The oligonucleotides are designed to have annealing sites which overlap a PCR primer annealing site. When the non-extendable oligonucleotide is bound, the PCR primer cannot bind and therefore a PCR product is not generated. When the non-extendable oligonucleotide is not bound, because of a mismatch, the primer-binding site is accessible and a PCR product is generated. See Cottrell, S E et al. A real-time PCR assay for DNA-methylation using methylation-specific blockers. Nuc Acids Res 2004, 32, 1.

MethylQuant is a technology that involves treatment of genomic DNA with sodium bisulfite followed by a PCR reaction. Sodium bisulfite treatment converts an unmethylated cytosine to uracil, while methylated cytosine residues remain unmodified. Quantification of the methylation status of a specific cytosine is performed by a methylation-specific real-time PCR reaction analyzed with a highly sensitive fluorescent stain for detecting dsDNA. One of the PCR primers is designed to have a 3′ end that discriminates between the bisulfite-converted uracil and the unmodified cytosine. The quantification is based on comparison of two PCRs performed with primer sets that amplify the target sequence either irrespective of methylation or in a methylation-specific manner. See Thomassin H. et al. MethylQuant: a sensitive method for quantifying methylation of specific cytosines within the genome. 2004. Nuc Acid Res 32, 21.

Enzymatic Regional Methylation Assay (ERMA) begins with sodium bisulfite-treated DNA in which unmethylated cytosine residues are converted to uracil residues. One then performs a PCR reaction amplifying a specific region of the DNA containing a potential methylation site. The PCR primers used in the reaction are designed to contain a GATC sequence, which is the recognition site for E. coli dam methyltransferase. After the PCR product is generated, it is treated with an E. coli cytosine methyltransferase, Sss1, which specifically methylates a cytosine in every CpG dinucleotide using a 3H-labeled methyl group donor. The incorporation of ³H-labeled methyl groups is proportional to the number of methylated CpG sites originally present in the template DNA. The PCR product and dam methyltransferase are then incubated with a ¹⁴C-labeled methyl group donor, which will label the GATC sequences in all PCR products. The E. coli dam methyltransferase is used as an internal control to standardize the amount of DNA that is analyzed, since all PCR products contain the GATC recognition sequence. The results are expressed as the ratio of the scintillation counting signals of both radioisotopes (³H/¹⁴C). See Galm et al. Enzymatic Regional Methylation Assay: A Novel Method to Quantify Regional CpG Methylation Density. Genome Research. Vol. 12, Issue 1, 153-157, January 2002

Ligase Chain Reaction (LCR) relies on DNA ligase to join adjacent oligonucleotides after they have annealed to a target DNA. The oligonucleotides are designed to be small and have a low annealing temperature, so they are destabilized by a single base mismatch. For methylation detection, a single base mismatch would arise from sodium bisulfite treatment of DNA, which converts an unmethylated cytosine to uracil, while methylated cytosine residues remain unaffected. A LCR to detect methylation requires two primer sets, one complementary to a bisulfite-modified cytosine in the DNA (converted to uracil) and another set complementary to a methylated cytosine in the DNA (resistant to bisulfite conversion). If there is a mismatch, the ligase reaction will not proceed and no product will be generated. One can visualize the ligated DNA product via gel electrophoresis and deduce the status of methylation.

The principle behind electrophoresis is the separation of nucleic acids via their size and charge. Many assays exist for detecting methylation and most rely on determining the presence or absence of a specific nucleic acid product. Gel electrophoresis is commonly used in a laboratory for this purpose.

One may use MALDI mass spectrometry in combination with a methylation detection assay to observe the size of a nucleic acid product. The principle behind mass spectrometry is the ionizing of nucleic acids and separating them according to their mass to charge ratio. Similar to electrophoresis, one can use mass spectrometry to detect a specific nucleic acid that was created in an experiment to determine methylation. See Tost, J. et al. Analysis and accurate quantification of CpG methylation by MALDI mass spectrometry. Nuc Acid Res, 2003, 31, 9

One form of chromatography, high performance liquid chromatography, is used to separate components of a mixture based on a variety of chemical interactions between a substance being analyzed and a chromatography column. DNA is first treated with sodium bisulfite, which converts an unmethylated cytosine to uracil, while methylated cytosine residues remain unaffected. One may amplify the region containing potential methylation sites via PCR and separate the products via denaturing high performance liquid chromatography (DHPLC). DHPLC has the resolution capabilities to distinguish between methylated (containing cytosine) and unmethylated (containing uracil) DNA sequences. See Deng, D. et al. Simultaneous detection of CpG methylation and single nucleotide polymorphism by denaturing high performance liquid chromatography. 2002 Nuc Acid Res, 30, 3.

Hybridization is a technique for detecting specific nucleic acid sequences that is based on the annealing of two complementary nucleic acid strands to form a double-stranded molecule. One example of the use of hybridization is a microarray assay to determine the methylation status of DNA. After sodium bisulfite treatment of DNA, which converts an unmethylated cytosine to uracil while methylated cytosine residues remain unaffected, oligonucleotides complementary to potential methylation sites can hybridize to the bisulfite-treated DNA. The oligonucleotides are designed to be complimentary to either sequence containing uracil or sequence containing cytosine, representing unmethylated and methylated DNA, respectively. Computer-based microarray technology can determine which oligonucleotides hybridize with the DNA sequence and one can deduce the methylation status of the DNA.

An additional method of determining the results after sodium bisulfite treatment would be to sequence the DNA to directly observe any bisulfite-modifications. Pyrosequencing technology is a method of sequencing-by-synthesis in real time. It is based on an indirect bioluminometric assay of the pyrophosphate (PPi) that is released from each deoxynucleotide (dNTP) upon DNA-chain elongation. This method presents a DNA template-primer complex with a dNTP in the presence of an exonuclease-deficient Klenow DNA polymerase. The four nucleotides are sequentially added to the reaction mix in a predetermined order. If the nucleotide is complementary to the template base and thus incorporated, PPi is released. The PPi and other reagents are used as a substrate in a luciferase reaction producing visible light that is detected by either a luminometer or a charge-coupled device. The light produced is proportional to the number of nucleotides added to the DNA primer and results in a peak indicating the number and type of nucleotide present in the form of a pyrogram. Pyrosequencing can exploit the sequence differences that arise following sodium bisulfite-conversion of DNA.

A variety of amplification techniques may be used in a reaction for creating distinguishable products. Some of these techniques employ PCR. Other suitable amplification methods include the ligase chain reaction (LCR) (Barringer et al, 1990), transcription amplification (Kwoh et al. 1989; WO88/10315), selective amplification of target polynucleotide sequences (U.S. Pat. No. 6,410,276), consensus sequence primed polymerase chain reaction (U.S. Pat. No. 4,437,975), arbitrarily primed polymerase chain reaction (WO90/06995), nucleic acid based sequence amplification (NASBA) (U.S. Pat. Nos. 5,409,818; 5,554,517; 6,063,603), nick displacement amplification (WO2004/067726).

Sequence variation that reflects the methylation status at CpG dinucleotides in the original genomic DNA offers two approaches to PCR primer design. In the first approach, the primers do not themselves “cover” or hybridize to any potential sites of DNA methylation; sequence variation at sites of differential methylation are located between the two primers. Such primers are used in bisulphite genomic sequencing, COBRA, Ms-SNuPE. In the second approach, the primers are designed to anneal specifically with either the methylated or unmethylated version of the converted sequence. If there is a sufficient region of complementarity, e.g., 12, 15, 18, or 20 nucleotides, to the target, then the primer may also contain additional nucleotide residues that do not interfere with hybridization but may be useful for other manipulations. Exemplary of such other residues may be sites for restriction endonuclease cleavage, for ligand binding or for factor binding or linkers or repeats. The oligonucleotide primers may or may not be such that they are specific for modified methylated residues

One way to distinguish between modified and unmodified DNA is to hybridize oligonucleotide primers which specifically bind to one form or the other of the DNA. After hybridization, an amplification reaction can be performed and amplification products assayed. The presence of an amplification product indicates that a sample hybridized to the primer. The specificity of the primer indicates whether the DNA had been modified or not, which in turn indicates whether the DNA had been methylated or not. For example, bisulfite ions modify non-methylated cytosine bases, changing them to uracil bases. Uracil bases hybridize to adenine bases under hybridization conditions. Thus an oligonucleotide primer which comprises adenine bases in place of guanine bases would hybridize to the bisulfite-modified DNA, whereas an oligonucleotide primer containing the guanine bases would hybridize to the non-modified (methylated) cytosine residues in the DNA. Amplification using a DNA polymerase and a second primer yield amplification products which can be readily observed. Such a method is termed MSP (Methylation Specific PCR; U.S. Pat. Nos. 5,786,146; 6,017,704; 6,200,756). The amplification products can be optionally hybridized to specific oligonucleotide probes which may also be specific for certain products. Alternatively, oligonucleotide probes can be used which will hybridize to amplification products from both modified and nonmodified DNA.

Another way to distinguish between modified and nonmodified DNA is to use oligonucleotide probes which may also be specific for certain products. Such probes can be hybridized directly to modified DNA or to amplification products of modified DNA. Oligonucleotide probes can be labeled using any detection system known in the art. These include but are not limited to fluorescent moieties, radioisotope labeled moieties, bioluminescent moieties, luminescent moieties, chemiluminescent moieties, enzymes, substrates, receptors, or ligands.

Still another way for the identification of methylated CpG dinucleotides utilizes the ability of the MBD domain of the McCP2 protein to selectively bind to methylated DNA sequences (Cross et al, 1994; Shiraishi et al, 1999). Restriction enconuclease digested genomic DNA is loaded onto expressed His-tagged methyl-CpG binding domain that is immobilized to a solid matrix and used for preparative column chromatography to isolate highly methylated DNA sequences.

Real time chemistry allows for the detection of PCR amplification during the early phases of the reactions, and makes quantitation of DNA and RNA easier and more precise. A few variations of the real-time PCR are known. They include the TaqMan™ system and Molecular Beacon™ system which have separate probes labeled with a fluorophore and a fuorescence quencher. In the Scorpion™ system the labeled probe in the form of a hairpin structure is linked to the primer.

DNA methylation analysis has been performed successfully with a number of techniques which include the MALDI-TOFF, MassARRAY, MethyLight, Quantitative analysis of ethylated alleles (QAMA), enzymatic regional methylation assay (ERMA), HeavyMethyl, QBSUPT, MS-SNuPE, MethylQuant, Quantitative PCR sequencing, and Oligonucleotide-based microarray systems.

The number of genes whose silencing is tested and/or detected can vary: one, two, three, four, five, or more genes can be tested and/or detected. In some cases at least two genes are selected. In other embodiments at least three genes are selected.

Testing can be performed diagnostically or in conjunction with a therapeutic regimen. Testing can be used to monitor efficacy of a therapeutic regimen, whether a chemotherapeutic agent or a biological agent, such as a polynucleotide. Testing can also be used to determine what therapeutic or preventive regimen to employ on a patient. Moreover, testing can be used to stratify patients into groups for testing agents and determining their efficacy on various groups of patients.

Test samples for diagnostic, prognostic, or personalized medicine uses can be obtained from surgical samples, such as biopsies or fine needle aspirates, from paraffin embedded colon, rectum, small intestinal, gastric, esophageal, bone marrow, breast, ovary, prostate, kidney, lung, brain on other organ tissues, from a body fluid such as blood, serum, lymph, cerebrospinal fluid, saliva, sputum, bronchial-lavage fluid, ductal fluids stool, urine, lymph nodes, or semen. Such sources are not meant to be exhaustive, but rather exemplary. A test sample obtainable from such specimens or fluids includes detached tumor cells or free nucleic acids that are released from dead or damaged tumor cells. Nucleic acids include RNA, genomic DNA, mitochondrial DNA, single or double stranded, and protein-associated nucleic acids. Any nucleic acid specimen in purified or non-purified form obtained from such specimen cell can be utilized as the starting nucleic acid or acids.

Demethylating agents can be contacted with cells in vitro or in vivo for the purpose of restoring normal gene expression to the cell. Suitable demethylating agents include, but are not limited to 5-aza-2′-deoxycytidine, 5-aza-cytidine, Zebularine, procaine, and L-ethionine. This reaction may be used for diagnosis, for determining predisposition, and for determining suitable therapeutic regimes. If the demethylating agent is used for treating colon, head and neck, esophageal, gastric, pancreatic, or liver cancers, expression or methylation can be tested of a gene selected from the group shown in Table 1.

An alternative way to restore epigenetically silenced gene expression is to introduce a non-methylated polynucleotide into a cell, so that it will be expressed in the cell. Various gene therapy vectors and vehicles are known in the art and any can be used as is suitable for a particular situation. Certain vectors are suitable for short term expression and certain vectors are suitable for prolonged expression. Certain vectors are trophic for certain organs and these can be used as is appropriate in the particular situation. Vectors may be viral or non-viral. The polynucleotide can, but need not, be contained in a vector, for example, a viral vector, and can be formulated, for example, in a matrix such as a liposome, microbubbles. The polynucleotide can be introduced into a cell by administering the polynucleotide to the subject such that it contacts the cell and is taken up by the cell and the encoded polypeptide expressed. Preferably the specific polynucleotide will be one which the patient has been tested for and been found to carry a silenced version. The polynucleotides for treating colon, head and neck, esophageal, gastric, pancreas, liver cancers will typically encode a gene selected from those shown in Table 1.

Cells exhibiting methylation silenced gene expression generally are contacted with the demethylating agent in vivo by administering the agent to a subject. Where convenient, the demethylating agent can be administered using, for example, a catheterization procedure, at or near the site of the cells exhibiting unregulated growth in the subject, or into a blood vessel in which the blood is flowing to the site of the cells. Similarly, where an organ, or portion thereof, to be treated can be isolated by a shunt procedure, the agent can be administered via the shunt, thus substantially providing the agent to the site containing the cells. The agent also can be administered systemically or via other routes known in the art.

The polynucleotide can include, in addition to polypeptide coding sequence, operatively linked transcriptional regulatory elements, translational regulatory elements, and the like, and can be in the form of a naked DNA molecule, which can be contained in a vector, or can be formulated in a matrix such as a liposome or microbubbles that facilitates entry of the polynucleotide into the particular cell. The term “operatively linked” refers to two or more molecules that are positioned with respect to each other such that they act as a single unit and effect a function attributable to one or both molecules or a combination thereof. A polynucleotide sequence encoding a desired polypeptide can be operatively linked to a regulatory element, in which case the regulatory element confers its regulatory effect on the polynucleotide similar to the way in which the regulatory element would affect a polynucleotide sequence with which it normally is associated with in a cell.

The polynucleotide encoding the desired polypeptide to be administered to a mammal or a human or to be contacted with a cell may contain a promoter sequence, which can provide constitutive or, if desired, inducible or tissue specific or developmental stage specific expression of the polynucleotide, a polyA recognition sequence, and a ribosome recognition site or internal ribosome entry site, or other regulatory elements such as an enhancer, which can be tissue specific. The vector also may contain elements required for replication in a prokaryotic or eukaryotic host system or both, as desired. Such vectors, which include plasmid vectors and viral vectors such as bacteriophage, baculovirus, retrovirus, lentivirus, adenovirus, vaccinia virus, semliki forest virus and adeno-associated virus vectors, are well known and can be purchased from a commercial source (Promega, Madison Wis.; Stratagene, La Jolla Calif.; GIBCO/BRL, Gaithersburg Md.) or can be constructed by one skilled in the art (see, for example, Meth. Enzymol., Vol. 185, Goeddel, ed. (Academic Press, Inc., 1990); Jolly, Canc. Gene Ther. 1:51-64, 1994; Flotte, J. Bioenerg. Biomemb. 25:37-42, 1993; Kirshenbaum et al., J. Clin. Invest. 92:381-387, 1993; each of which is incorporated herein by reference).

A tetracycline (tet) inducible promoter can be used for driving expression of a polynucleotide encoding a desired polypeptide. Upon administration of tetracycline, or a tetracycline analog, to a subject containing a polynucleotide operatively linked to a tet inducible promoter, expression of the encoded polypeptide is induced. The polynucleotide alternatively can be operatively linked to tissue specific regulatory element, for example, a liver cell specific regulatory element such as an α.-fetoprotein promoter (Kanai et al., Cancer Res. 57:461-465, 1997; He et al., J. Exp. Clin. Cancer Res. 19:183-187, 2000) or an albumin promoter (Power et al., Biochem. Biophys. Res. Comm. 203:1447-1456, 1994; Kuriyama et al., Int. J. Cancer 71:470-475, 1997); a muscle cell specific regulatory element such as a myoglobin promoter (Devlin et al., J. Biol. Chem. 264:13896-13901, 1989; Yan et al., J. Biol. Chem. 276:17361-17366, 2001); a prostate cell specific regulatory element such as the PSA promoter (Schuur et al., J. Biol. Chem. 271:7043-7051, 1996; Latham et al., Cancer Res. 60:334-341, 2000); a pancreatic cell specific regulatory element such as the elastase promoter (Omitz et al., Nature 313:600-602, 1985; Swift et al., Genes Devel. 3:687-696, 1989); a leukocyte specific regulatory element such as the leukosialin (CD43) promoter (Shelley et al., Biochem. J. 270:569-576, 1990; Kudo and Fukuda, J. Biol. Chem. 270:13298-13302, 1995); or the like, such that expression of the polypeptide is restricted to particular cell in an individual, or to particular cells in a mixed population of cells in culture, for example, an organ culture. Regulatory elements, including tissue specific regulatory elements, many of which are commercially available, are well known in the art (see, for example, InvivoGen; San Diego Calif.).

Viral expression vectors can be used for introducing a polynucleotide into a cell, particularly a cell in a subject. Viral vectors provide the advantage that they can infect host cells with relatively high efficiency and can infect specific cell types. For example, a polynucleotide encoding a desired polypeptide can be cloned into a baculovirus vector, which then can be used to infect an insect host cell, thereby providing a means to produce large amounts of the encoded polypeptide. Viral vectors have been developed for use in particular host systems, particularly mammalian systems and include, for example, retroviral vectors, other lentivirus vectors such as those based on the human immunodeficiency virus (HIV), adenovirus vectors, adeno-associated virus vectors, herpesvirus vectors, hepatitis virus vectors, vaccinia virus vectors, and the like (see Miller and Rosman, BioTechniques 7:980-990, 1992; Anderson et al., Nature 392:25-30 Suppl., 1998; Verma and Somia, Nature 389:239-242, 1997; Wilson, New Engl. J. Med. 334:1185-1187 (1996), each of which is incorporated herein by reference).

A polynucleotide, which can optionally be contained in a vector, can be introduced into a cell by any of a variety of methods known in the art (Sambrook et al., supra, 1989; Ausubel et al., Current Protocols in Molecular Biology, John Wiley and Sons, Baltimore, Md. (1987, and supplements through 1995), each of which is incorporated herein by reference). Such methods include, for example, transfection, lipofection, microinjection, electroporation and, with viral vectors, infection; and can include the use of liposomes, microemulsions or the like, which can facilitate introduction of the polynucleotide into the cell and can protect the polynucleotide from degradation prior to its introduction into the cell. A particularly useful method comprises incorporating the polynucleotide into microbubbles, which can be injected into the circulation. An ultrasound source can be positioned such that ultrasound is transmitted to the tumor, wherein circulating microbubbles containing the polynucleotide are disrupted at the site of the tumor due to the ultrasound, thus providing the polynucleotide at the site of the cancer. The selection of a particular method will depend, for example, on the cell into which the polynucleotide is to be introduced, as well as whether the cell is in culture or in situ in a body.

Introduction of a polynucleotide into a cell by infection with a viral vector can efficiently introduce the nucleic acid molecule into a cell. Moreover, viruses are very specialized and can be selected as vectors based on an ability to infect and propagate in one or a few specific cell types. Thus, their natural specificity can be used to target the nucleic acid molecule contained in the vector to specific cell types. A vector based on an HIV can be used to infect T cells, a vector based on an adenovirus can be used, for example, to infect respiratory epithelial cells, a vector based on a herpesvirus can be used to infect neuronal cells, and the like. Other vectors, such as adeno-associated viruses can have greater host cell range and, therefore, can be used to infect various cell types, although viral or non-viral vectors also can be modified with specific receptors or ligands to alter target specificity through receptor mediated events. A polynucleotide of the invention, or a vector containing the polynucleotide can be contained in a cell, for example, a host cell, which allows propagation of a vector containing the polynucleotide, or a helper cell, which allows packaging of a viral vector containing the polynucleotide. The polynucleotide can be transiently contained in the cell, or can be stably maintained due, for example, to integration into the cell genome.

A polypeptide encoded by a gene disclosed in Table 1 can be administered directly to the site of a cell exhibiting unregulated growth in the subject. The polypeptide can be produced and isolated, and formulated as desired, using methods as disclosed herein, and can be contacted with the cell such that the polypeptide can cross the cell membrane of the target cells. The polypeptide may be provided as part of a fusion protein, which includes a peptide or polypeptide component that facilitates transport across cell membranes. For example, a human immunodeficiency virus (HIV) TAT protein transduction domain or a nuclear localization domain may be fused to the marker of interest. The administered polypeptide can be formulated in a matrix that facilitates entry of the polypeptide into a cell.

While particular polynucleotide and polypeptide sequences are mentioned here as representative of known genes and proteins, those of skill in the art will understand that the sequences in the databases represent the sequences present in particular individuals. Any allelic sequences from other individuals can be used as well. These typically vary from the disclosed sequences at 1-10 residues, at 1-5 residues, or at 1-3 residues. Moreover, the allelic sequences are typically at least 95, 96, 97, 98, or 99% identical to the database sequence, as measured using an algorithm such as the BLAST homology tools.

An agent such as a demethylating agent, a polynucleotide, or a polypeptide is typically formulated in a composition suitable for administration to the subject. Thus, the invention provides compositions containing an agent that is useful for restoring regulated growth to a cell exhibiting unregulated growth due to methylation silenced transcription of one or more genes. The agents are useful as medicaments for treating a subject suffering from a pathological condition associated with such unregulated growth. Such medicaments generally include a carrier. Acceptable carriers are well known in the art and include, for example, aqueous solutions such as water or physiologically buffered saline or other solvents or vehicles such as glycols, glycerol, oils such as olive oil or injectable organic esters. An acceptable carrier can contain physiologically acceptable compounds that act, for example, to stabilize or to increase the absorption of the conjugate. Such physiologically acceptable compounds include, for example, carbohydrates, such as glucose, sucrose or dextrans, antioxidants, such as ascorbic acid or glutathione, chelating agents, low molecular weight proteins or other stabilizers or excipients. One skilled in the art would know or readily be able to determine an acceptable carrier, including a physiologically acceptable compound. The nature of the carrier depends on the physico-chemical characteristics of the therapeutic agent and on the route of administration of the composition. Administration of therapeutic agents or medicaments can be by the oral route or parenterally such as intravenously, intramuscularly, subcutaneously, transdermally, intranasally, intrabronchially, vaginally, rectally, intratumorally, or other such method known in the art. The pharmaceutical composition also can contain one more additional therapeutic agents.

The therapeutic agents can be incorporated within an encapsulating material such as into an oil-in-water emulsion, a microemulsion, micelle, mixed micelle, liposome, microsphere, microbubbles or other polymer matrix (see, for example, Gregoriadis, Liposome Technology, Vol. 1 (CRC Press, Boca Raton, Fla. 1984); Fraley, et al., Trends Biochem. Sci., 6:77 (1981), each of which is incorporated herein by reference). Liposomes, for example, which consist of phospholipids or other lipids, are nontoxic, physiologically acceptable and metabolizable carriers that are relatively simple to make and administer. “Stealth” liposomes (see, for example, U.S. Pat. Nos. 5,882,679; 5,395,619; and 5,225,212, each of which is incorporated herein by reference) are an example of such encapsulating materials particularly useful for preparing a composition useful in a method of the invention, and other “masked” liposomes similarly can be used, such liposomes extending the time that the therapeutic agent remain in the circulation. Cationic liposomes, for example, also can be modified with specific receptors or ligands (Morishita et al., J. Clin. Invest., 91:2580-2585 (1993), which is incorporated herein by reference). In addition, a polynucleotide agent can be introduced into a cell using, for example, adenovirus-polylysine DNA complexes (see, for example, Michael et al., J. Biol. Chem. 268:6866-6869 (1993), which is incorporated herein by reference).

The route of administration of the composition containing the therapeutic agent will depend, in part, on the chemical structure of the molecule. Polypeptides and polynucleotides, for example, are not efficiently delivered orally because they can be degraded in the digestive tract. However, methods for chemically modifying polypeptides, for example, to render them less susceptible to degradation by endogenous proteases or more absorbable through the alimentary tract may be used (see, for example, Blondelle et al., supra, 1995; Ecker and Crook, supra, 1995).

The total amount of an agent to be administered in practicing a method of the invention can be administered to a subject as a single dose, either as a bolus or by infusion over a relatively short period of time, or can be administered using a fractionated treatment protocol, in which multiple doses are administered over a prolonged period of time. One skilled in the art would know that the amount of the composition to treat a pathologic condition in a subject depends on many factors including the age and general health of the subject as well as the route of administration and the number of treatments to be administered. In view of these factors, the skilled artisan would adjust the particular dose as necessary. In general, the formulation of the composition and the routes and frequency of administration are determined, initially, using Phase I and Phase II clinical trials.

The composition can be formulated for oral formulation, such as a tablet, or a solution or suspension form; or can comprise an admixture with an organic or inorganic carrier or excipient suitable for enteral or parenteral applications, and can be compounded, for example, with the usual non-toxic, pharmaceutically acceptable carriers for tablets, pellets, capsules, suppositories, solutions, emulsions, suspensions, or other form suitable for use. The carriers, in addition to those disclosed above, can include glucose, lactose, mannose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening or coloring agents and perfumes can be used, for example a stabilizing dry agent such as triulose (see, for example, U.S. Pat. No. 5,314,695).

Although diagnostic and prognostic accuracy and sensitivity may be achieved by using a combination of markers, such as 5 or 6 markers, or 9 or 10 markers, or 14 or 15 markers, practical considerations may dictate use of smaller combinations. Any combination of markers for a specific cancer may be used which comprises 2, 3, 4, or 5 markers. Combinations of 2, 3, 4, or 5 markers can be readily envisioned given the specific disclosures of individual markers provided herein.

The level of methylation of the differentially methylated GpG islands can provide a variety of information about the disease or cancer. It can be used to diagnose pre-cancer or cancer in the individual. Pre-cancer or cancer precursor is a very early stage of cancer which is found in the innermost (luminal) layer of the colon. It is sometimes referred to as superficial cancer. Alternatively, it can be used to predict the course of the disease or cancer in the individual or to predict the suspectibility to disease or cancer or to stage the progression of the disease or cancer in the individual. It can help to predict the likelihood of overall survival or predict the likelihood of reoccurrence of disease or cancer and to determine the effectiveness of a treatment course undergone by the individual. Increase or decrease of methylation levels in comparison with reference level and alterations in the increase/decrease when detected provide useful prognostic and diagnostic value.

The prognostic methods can be used to identify patients with adenomas that are likely to progress to carcinomas. Such a prediction can be made on the basis of epigenetic silencing of at least one of the genes identified in Table 1 in an adenoma relative to normal tissue. Such patients can be offered additional appropriate therapeutic or preventative options, including endoscopic polypectomy or resection, and when indicated, surgical procedures, chemotherapy, radiation, biological response modifiers, or other therapies. Such patients may also receive recommendations for further diagnostic or monitoring procedures, including but not limited to increased frequency of colonoscopy, sigmoidoscopy, virtual colonoscopy, video capsule endoscopy, PET-CT, molecular imaging, or other imaging techniques.

A therapeutic strategy for treating a cancer patient can be selected based on reactivation of epigenetically silenced genes. First a gene selected from those listed in Table 1 is identified whose expression in cancer cells of the patient is reactivated by a demethylating agent or epigenetically silenced. A treatment which increases the expression of the gene is then selected. Such a treatment can comprise administration of a reactivating agent or a polynucleotide. A polypeptide can alternatively be administered.

Kits according to the present invention are assemblages of reagents for testing methylation. They are typically in a package which contains all elements, optionally including instructions. The package may be divided so that components are not mixed until desired. Components may be in different physical states. For example, some components may be lyophilized and some in aqueous solution. Some may be frozen. Individual components may be separately packaged within the kit. The kit may contain reagents, as described above for differentially modifying methylated and non-methylated cytosine residues. Desirably the kit will contain oligonucleotide primers which specifically hybridize to regions within 1 kb of the transcription start sites of the genes/markers identified in the attached Table 1. Typically the kit will contain both a forward and a reverse primer for a single gene or marker. If there is a sufficient region of complementarity, e.g., 12, 15, 18, or 20 nucleotides, then the primer may also contain additional nucleotide residues that do not interfere with hybridization but may be useful for other manipulations. Exemplary of such other residues may be sites for restriction endonuclease cleavage, for ligand binding or for factor binding or linkers or repeats. The oligonucleotide primers may or may not be such that they are specific for modified methylated residues. The kit may optionally contain oligonucleotide probes. The probes may be specific for sequences containing modified methylated residues or for sequences containing non-methylated residues. The kit may optionally contain reagents for modifying methylated cytosine residues. The kit may also contain components for performing amplification, such as a DNA polymerase and deoxyribonucleotides. Means of detection may also be provided in the kit, including detectable labels on primers or probes. Kits may also contain reagents for detecting gene expression for one or more of the markers of the present invention (Table 1). Such reagents may include probes, primers, or antibodies, for example. In the case of enzymes or ligands, substrates or binding partners may be sued to assess the presence of the marker.

In one aspect of this embodiment, the gene is contacted with hydrazine, which modifies cytosine residues, but not methylated cytosine residues, then the hydrazine treated gene sequence is contacted with a reagent such as piperidine, which cleaves the nucleic acid molecule at hydrazine modified cytosine residues, thereby generating a product comprising fragments. By separating the fragments according to molecular weight, using, for example, an electrophoretic, chromatographic, or mass spectrographic method, and comparing the separation pattern with that of a similarly treated corresponding non-methylated gene sequence, gaps are apparent at positions in the test gene contained methylated cytosine residues. As such, the presence of gaps is indicative of methylation of a cytosine residue in the CpG dinucleotide in the target gene of the test cell.

Bisulfite ions, for example, sodium bisulfite, convert non-methylated cytosine residues to bisulfite modified cytosine residues. The bisulfite ion treated gene sequence can be exposed to alkaline conditions, which convert bisulfite modified cytosine residues to uracil residues. Sodium bisulfite reacts readily with the 5,6-double bond of cytosine (but poorly with methylated cytosine) to form a sulfonated cytosine reaction intermediate that is susceptible to deamination, giving rise to a sulfonated uracil. The sulfonate group can be removed by exposure to alkaline conditions, resulting in the formation of uracil. The DNA can be amplified, for example, by PCR, and sequenced to determine whether CpG sites are methylated in the DNA of the sample. Uracil is recognized as a thymine by Taq polymerase and, upon PCR, the resultant product contains cytosine only at the position where 5-methylcytosine was present in the starting template DNA. One can compare the amount or distribution of uracil residues in the bisulfite ion treated gene sequence of the test cell with a similarly treated corresponding non-methylated gene sequence. A decrease in the amount or distribution of uracil residues in the gene from the test cell indicates methylation of cytosine residues in CpG dinucleotides in the gene of the test cell. The amount or distribution of uracil residues also can be detected by contacting the bisulfite ion treated target gene sequence, following exposure to alkaline conditions, with an oligonucleotide that selectively hybridizes to a nucleotide sequence of the target gene that either contains uracil residues or that lacks uracil residues, but not both, and detecting selective hybridization (or the absence thereof) of the oligonucleotide.

Test compounds can be tested for their potential to treat cancer. Cancer cells for testing can be selected from the group consisting of prostate, lung, breast, and colon cancer. Expression of a gene selected from those listed in Table 1 is determined and if it is increased by the compound in the cell or if methylation of the gene is decreased by the compound in the cell, one can identify it as having potential as a treatment for cancer.

Alternatively such tests can be used to determine an esophageal, head and neck, gastric, small intestinal, pancreas, liver cancer patient's response to a chemotherapeutic agent. The patient can be treated with a chemotherapeutic agent. If expression of a gene selected from those listed in Table 1 is increased by the compound in cancer cells or if methylation of the gene is decreased by the compound in cancer cells it can be selected as useful for treatment of the patient.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### EXAMPLES

### Example 1

### Materials and Methods

Cell culture and treatment. HCT116 cells and isogenic genetic knockout derivatives were maintained as previously described (Rhee et al¹). For drug treatments, log phase HCT116 cells were cultured in McCoys 5A media (Invitrogen) containing 10% BCS and 1× penicillin/streptomycin with 5 μM 5-aza-deoxycytidine (DAC) (Sigma; stock solution: 1 mM in PBS) for 96 hours, replacing media and DAC every 24 hours. Cell treatment with 300 nM Trichostatin A (Sigma; stock solution: 1.5 mM dissolved in Ethanol) was performed for 18 hours. Control cells underwent mock treatment in parallel with addition of equal volume of PBS without drugs.

Microarray analysis. Total RNA was harvested from log phase cells using the Qiagen kit according to the manufacturers instructions, including a DNAase step. RNA was quantified using the Nanoprop ND-100 followed by quality assessment with 2100 Bioanalyzer (Agilent Technologies). RNA concentrations for individual samples were greater than 200 ng/ul, with 28 s/18 s ratios greater than 2.2 and RNA integrity numbers of 10. Sample amplification and labeling procedures were carried out using the Low RNA Input Fluorescent Linear Amplification Kit (Agilent Technologies) according to the manufacturers instructions. The labeled cRNA was purified using the RNeasy mini kit (Qiagen) and quantified. RNA spike-in controls (Agilent Technologies) were added to RNA samples before amplification. 0.75 microgram of samples labeled with Cy3 or Cy5 were mixed with control targets (Agilent Technologies), assembled on Oligo Microarray, hybridized, and processed according to the Agilent microarray protocol. Scanning was performed with the Agilent G2565BA microarray scanner under default settings recommended by Agilent Technologies.

Data analysis. All arrays were subject to quality checks recommended by the manufacturer. Images were visually inspected for artifacts and distributions of signal and background intensity of both red and green channels were examined to identify anomalous arrays. No irregularities were observed, and all arrays were retained and used. All calculations were performed using the R statistical computing platform (Ihaka and Gentleman) and packages from Bioconductor bioinformatics software project (Gentleman et al.). The log ratio of red signal to green signal was calculated after background-subtraction and LoEss normalization as implemented in the limma package from Bioconductor (Smyth et al. 1; Smyth et al. 2). Individual arrays were scaled to have the same inter-quartile range (75^{th} percentile-25^{th} percentile) Log fold changes were averaged over dye-swap replicate microarrays to produce a single set of expression values for each condition.

Methylation and gene expression analysis. RNA was isolated with TRIzol™ Reagent (Invitrogen) according to the manufacturer's instructions. For reverse transcription-PCR (RT-PCR), 1 μg of total RNA was reverse transcribed by using Ready-To-Go™ You-Prime First-Strand Beads (Amersham Biosciences) with addition of random hexamers (0.2 μg per reaction). For RT-primer design we used Primer3 (at the URL address: http file type, domain name Frodo, wi.mit.edu directory, document cgi-bin/primer3/primer3_www.cgi). For MSP analysis, DNA was extracted following a standard phenol-chloroform extraction method. Bisulfite modification of genomic DNA was carried out using the EZ DNA methylation Kit™ (Zymo Research). Primer sequences specific for the unmethylated and methylated promotor sequences were designed using MSPPrimer (at the URL address: http file type, www server, domain name mspprimer.org). MSP was performed as previously described (Herman et al.). All PCR products (15 μl of 50 μl total volume for RT-PCR and 7.5 μl of 25 μl total volume for MSP) were loaded directly onto 2% agarose gels containing GelStar™ Nucleic Acid Gel Stain (Cambrex Bio Science) and visualized under ultraviolet illumination. Primer sequences and conditions for MSP, bisulfite sequencing, and RT-PCR are available upon request.

Colony Formation Assay. One million HCT116, RKO, or DLD1 cells were plated in 6-well dishes (Falcon) and transfected with 5 μg of plasmid (pIRES-Neo3; Invitrogen) using Lipofectamine 2000 according to the manufacturer's instructions. Following a 24 hour recovery period, selection in 5 mg/ml Hygromycin containing complete medium was performed for 10 days. Staining, visualization and counting of triplicate wells were performed as previously described (Ting et al.).

### Example 2

### Results

In humans, a majority of the 3×10⁷ 5-methylcytosine nucleotides are embedded within repeat-rich DNA located outside of genes (Bestor et al.). However, CpG rich islands within the promoter regions of approximately 45% of genes remain primarily methylation free in normal somatic cells (Antequera and Bird). Some predictions suggest there may be hundreds of tumor suppressor genes with aberrant CpG island hypermethylation and transcriptional repression in human tumors (Costello et al., Suzuki et al.). Most, including functionally important genes, remain unidentified. Multiple approaches to screen for such genes have appeared including searches for hypermethylated loci in defined chromosomal regions (Wales et al.), methylation based screens of CpG island subsets (Hu et al., Weber et al.), gene expression profiling (Gius et al.; Paz et al.), and methylation sensitive restriction enzyme dependent genomic screens (Toyota et al., Keshet et al.; Ushijima). Each approach has limitations due to either inadequate genome coverage, as in candidate gene searches, or high false positive rates inherent to genome wide screens. Many of these studies have identified only a handful of new candidate hypermethylated genes.

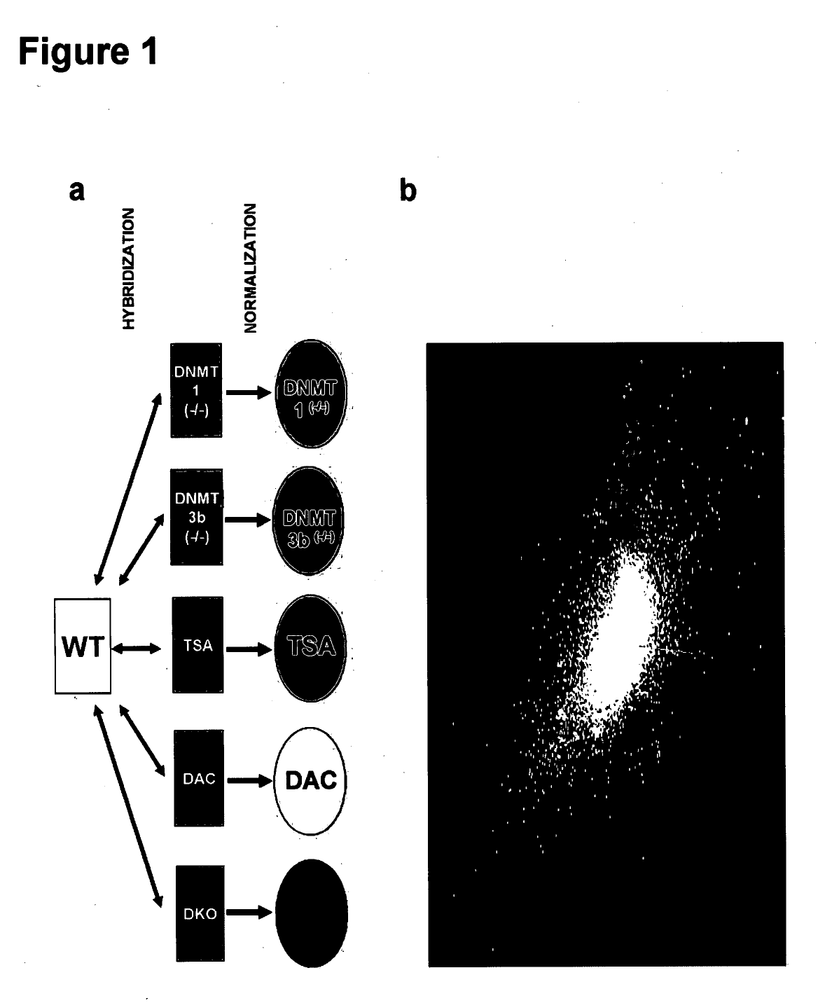

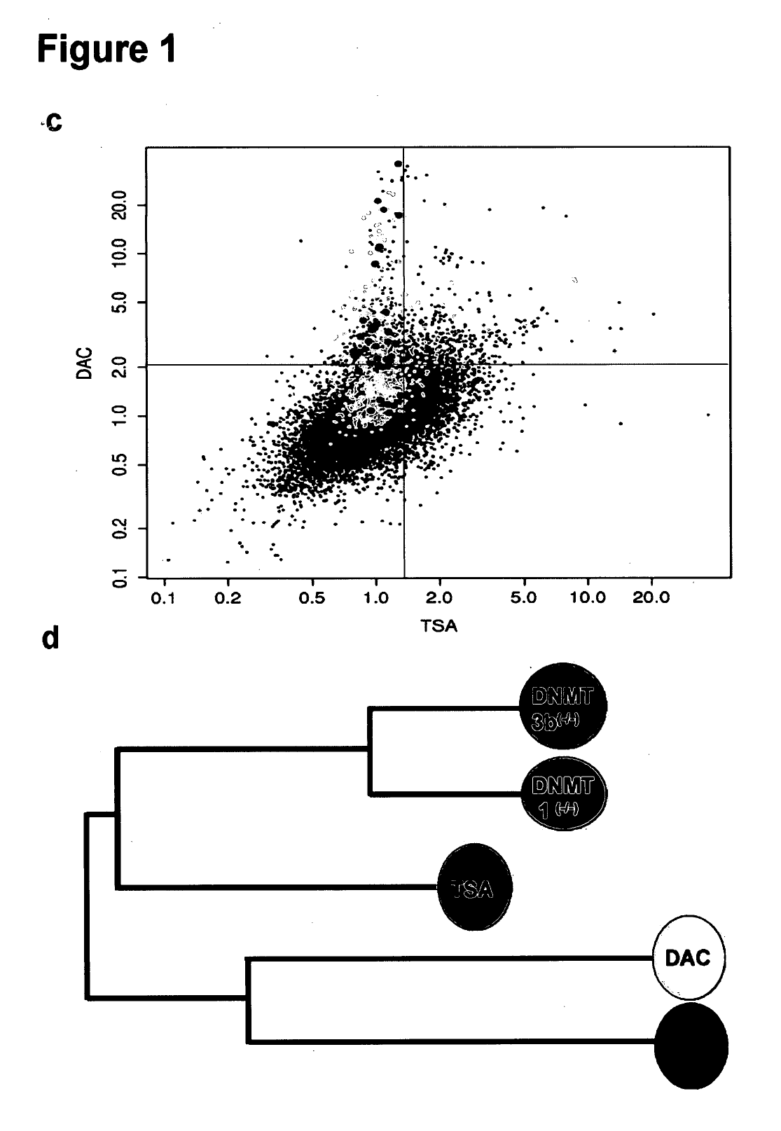

Here, we describe a microarray based gene expression screen, using whole human transcriptome arrays, for identifying genes silenced by promoter hypermethylation. We derive our approach by first comparing wild type HCT116 colon cancer cells with isogenic partner cells carrying genetic deletions of the major human DNA methyltransferases (Rhee et al. 2). Importantly, only in the DNMT1^{-/-}DNMT3b^{-/-} knockout (DKO) HCT116 cells, which have virtually complete loss of global 5-methylcytosine, do all individually examined hypermethylated genes undergo promoter demethylation with concomitant gene re-expression (Rhee et al., Suzuki et al 2, Akiyama et al, Toyota et al.). Accounting for the fact that densely hypermethylated genes have little or no basal expression and may produce low numbers of transcripts upon re-expression (FIG. 1A; Suzuki et al.), we detect a unique spike of hundreds of genes re-expressed in the DKO cells (FIG. 1B).

We next compared the genetic approach to a pharmacologic approach which might be used with any cancer cell type since targeted gene disruption of DNMTs is not feasible in most cell lines. For densely hypermethylated CpG island-containing genes, the DNA demethylating agent 5-deoxyazacytidine (DAC) and the class I and II HDAC inhibitor, trichostatin A (TSA), synergize for re-expression but TSA treatment alone is unable to induce gene expression (Suzuki et al., Cameron et al.). In a past microrarray study, we screened approximately 10,000 genes by treating cells with both agents together, enriched for a small subset of re-expressed genes with a subtraction protocol, and verified that truly hypermethylated candidate genes were re-expressed with DAC, but not with TSA alone, while genes re-expressed with TSA were not hypermethylated (Suzuki et al.). Here, we treated HCT116 cells with either agent alone, and identified a zone in which gene expression did not change with TSA (<1.4 fold up or down) and looked at DAC response within this region. We identify a distinct spike of DAC induced expression (>2 fold) for genes in this zone which highly overlaps (compare FIG. 1C yellow spots with black) with the spike of increased genes in the DKO cells. Taken together these data indicate a direct relationship between genetically and pharmacologically induced demethylation dependent gene expression when failure to respond to HDAC inhibition is taken into account (FIG. 1D).

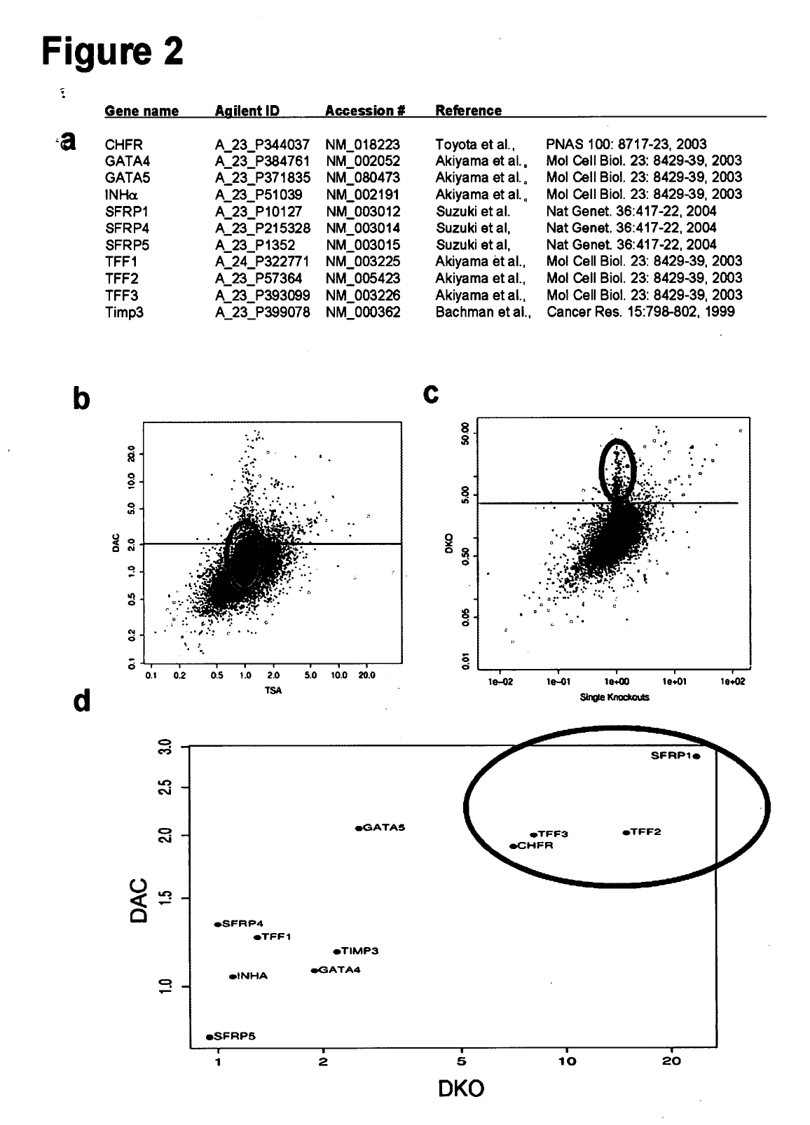

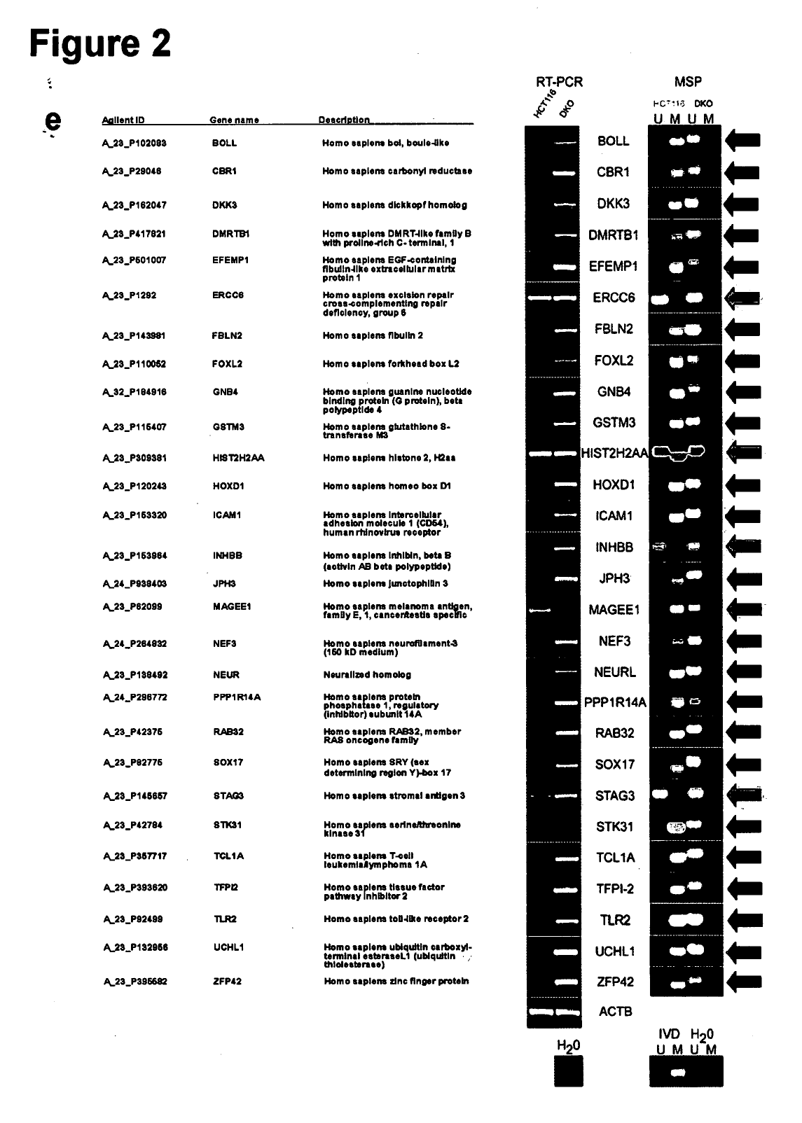

How many genes identified by our approach are truly CpG island hypermethylated genes and how efficiently does our search identify them? To begin addressing these issues, we first asked how 11 genes (FIG. 2a) known to be hypermethylated and completely silenced in HCT116 cells behaved on the microarrays. By RT-PCR analysis, each gene is known to be re-expressed in both DAC-treated and DKO cells (Akiyama et al.; Toyota et al., Rhee et al²). As predicted, all of these “guide” genes remained within the TSA non-responsive zone (FIG. 2B). Interestingly, these genes displayed a bimodal distribution of expression in both DAC treated and DKO cells (FIG. 2B-2D). Of the eleven tested guide genes, four (SFRP1, TFF2, TFF3, and CHFR) demonstrated re-expression responses near the top of both DKO and DAC expression spikes while the others did not increase, or minimally so (FIG. 2D).

We first examined whether the four top tier guide genes might aid in the identification of hypermethylated cancer genes. We matched 220 spots, containing 180 known genes, with characteristics of the top four guide genes, including no expression in mock treated HCT 116 cells, increases of >2 fold in DKO cells, >2 fold with DAC treatment, and failure to increase with TSA (<1.4 fold). From these genes we randomly selected a subset of 28 for experimental verification of both expression and methylation. For 27 of the 28, their gene expression increases spanned throughout the DAC spike (FIG. 1C green spots). One gene (JPH3) was found, retrospectively, to have a DAC response falling in the lower TSA negative zone (green spot with lowest intensity FIG. 1c) but was retained to test this region.

Results with these 28 test genes indicates an extraordinary efficiency for our screening approach. Twenty three of the 28 genes (82%), including JPH3, proved to be CpG hypermethylated and silenced in the wild type HCT116 cells. By sensitive RT-PCR analysis, these 23 genes were not basally expressed in HCT116 cells, but were distinctly re-expressed in demethylated DKO cells (FIG. 2E). In perfect concordance with these data, using the sensitive methylation specific PCR assay (MSP; Herman et al.), which specifically identifies methylated versus unmethylated sequences within CpG islands, we found 23 genes harboring signal only for methylated sequences in wildtype HCT116 cells and only unmethylated signals in DKO cells (FIG. 2E). Of the 5 false positive genes (FIG. 2E and FIG. 1C red spots), a range of results contributed including two genes that were unmethylated and basally expressed to a gene that was unmethylated, not basally expressed, and re-expressed in DKO cells. Failure to have proper annotation in the database for the true start site of the gene, and thus methylation analysis of the wrong CpG island region, could account for this latter result.

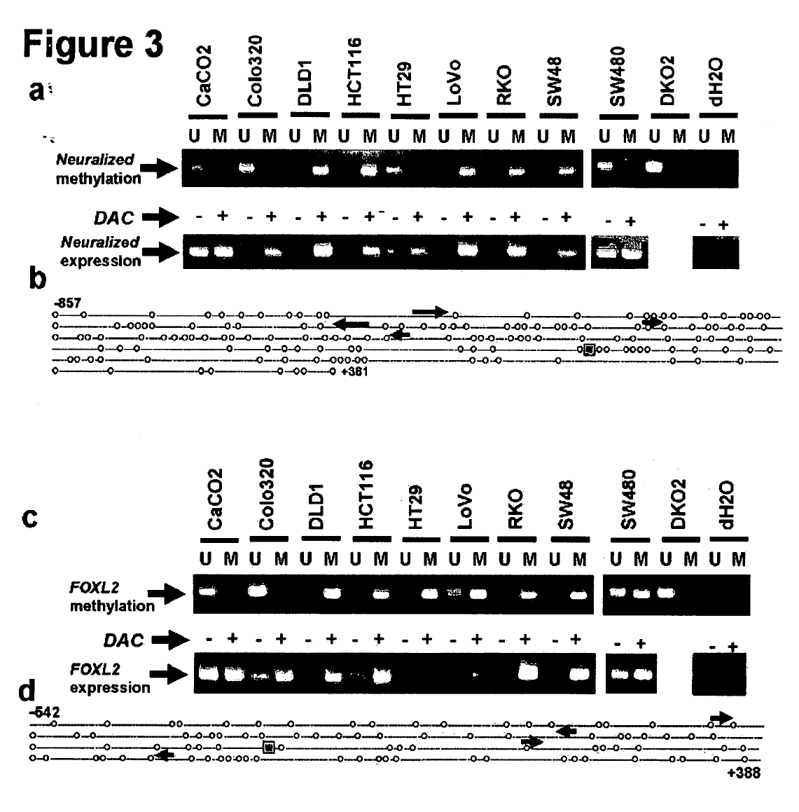

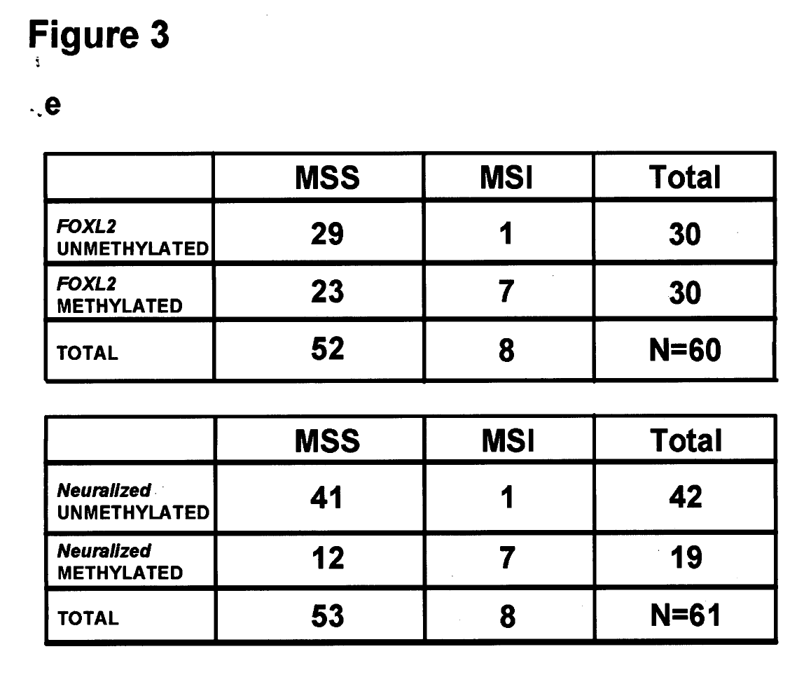

We next tested two of the 23 verified genes for their likely importance in primary colon cancers. One, the Neuralized gene (NEURL), is located in a chromosome region with high deletion frequency in brain tumors (Nakamura et al.) and its product has been identified as a ubiquitin ligase required for Notch ligand turnover (Pavlopoulos et al.; Deblandre et al.; Lai et al.). Activation of this key developmental pathway influences cell fate determination in flies and vertebrates (van Es et al.; Fre et al.) and activation of Notch, through unknown mechanisms, is thought to play an inhibitory role in normal differentiation during colorectal cancer (Radtke and Clevers). The second gene, FOXL2 belongs to the forkhead domain containing family of transcription factors implicated in diverse processes including establishing and maintaining differentiation programs (Lehmann et al.). Intriguingly, this gene is essential for proper ovarian development (Uda et al.) and germline mutations in humans lead to a plethora of craniofacial anomalies and premature ovarian failure (Crisponi et al.). We find, for the first time, that these genes are frequently DNA hypermethylated in a panel of colorectal cell lines (5 of 9 cell lines for Neuralized and 7 of 9 for FOXL2; FIGS. 3A and 3C) and bisulfite sequencing revealed methylation of all CpG residues in the central CpG island regions of both genes in HCT116 and RKO cell lines, with virtually complete demethylation in DKO cells (FIGS. 3B and 3C). For both genes, this hypermethylation perfectly correlated with loss of basal expression and ability to re-express the genes with DAC treatment (FIGS. 3A and 3C). Importantly, promoter methylation of both genes is absent in normal human colon or rectum suggesting that hypermethylation arose as a cancer specific phenomenon (FIGS. 3B and 3D).

Remarkably, the frequency for hypermethylation of the FOXL2 and Neuralized genes not only extends to primary human colon tumors but in a very important context to colon cancer biology. Nearly 1 in eight colorectal cancers, predominantly those from the right side of the colon, harbor a defect in mismatch repair capacity (Ionov et al., Parsons et al.) due to inactivation of MLH1 by genetic (Leach et al.) or epigenetic mechanisms (Herman et al.) and such tumors have a marked propensity for hypermethylating gene promoters (Toyota et al.²). The hypermethylation patterns of FOXL2 and, especially, Neuralized, aggregate with these tumor types not only among the colon cancer cell lines (HCT116, DLD1, LoVo, RKO and SW48), but also when analyzed in a series of primary human colon cancers (FIG. 3E). Our studies suggest that epigenetic inactivation of FOXL2 and Neuralized may belong to the important hypermethylator, or “CIMP,” phenotype described for colorectal tumors (Toyota et al.).

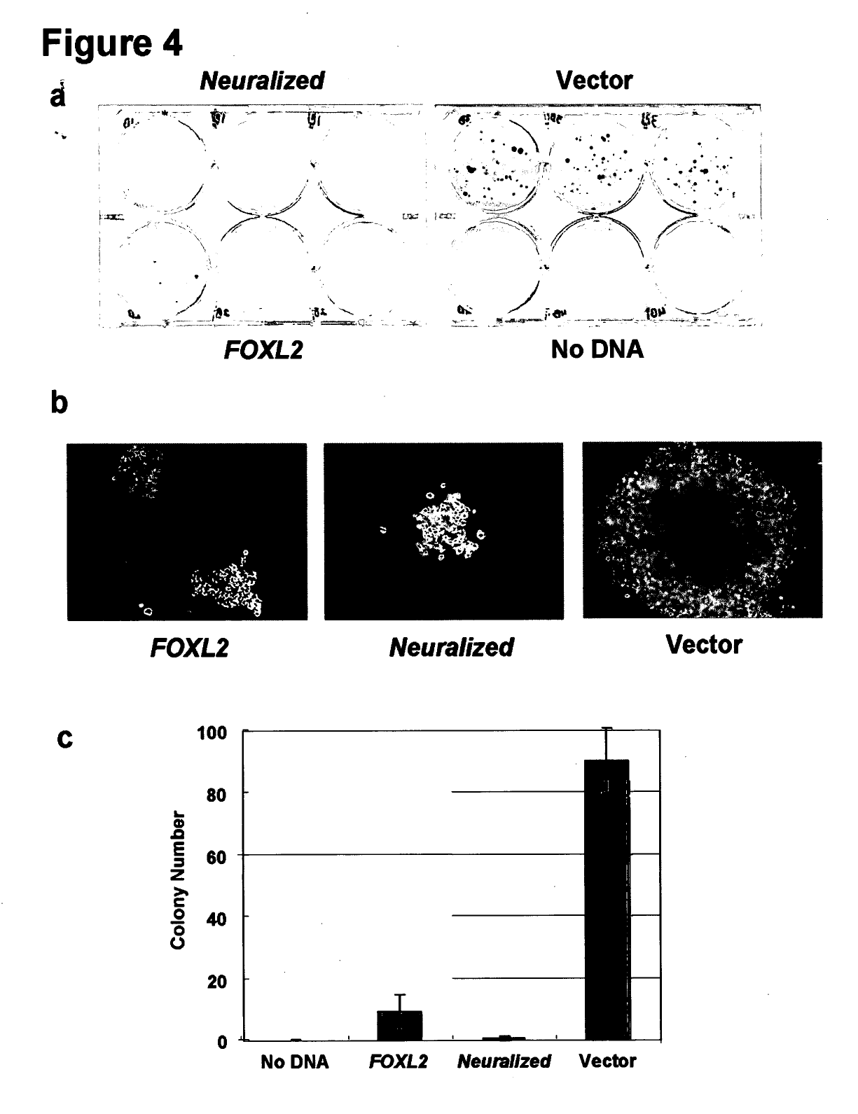

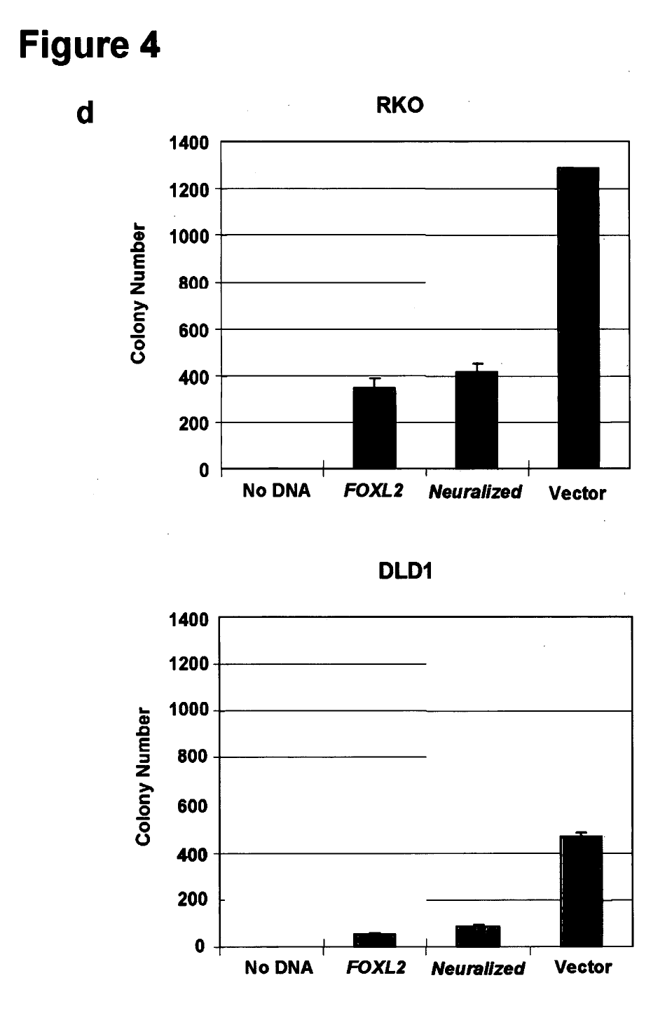

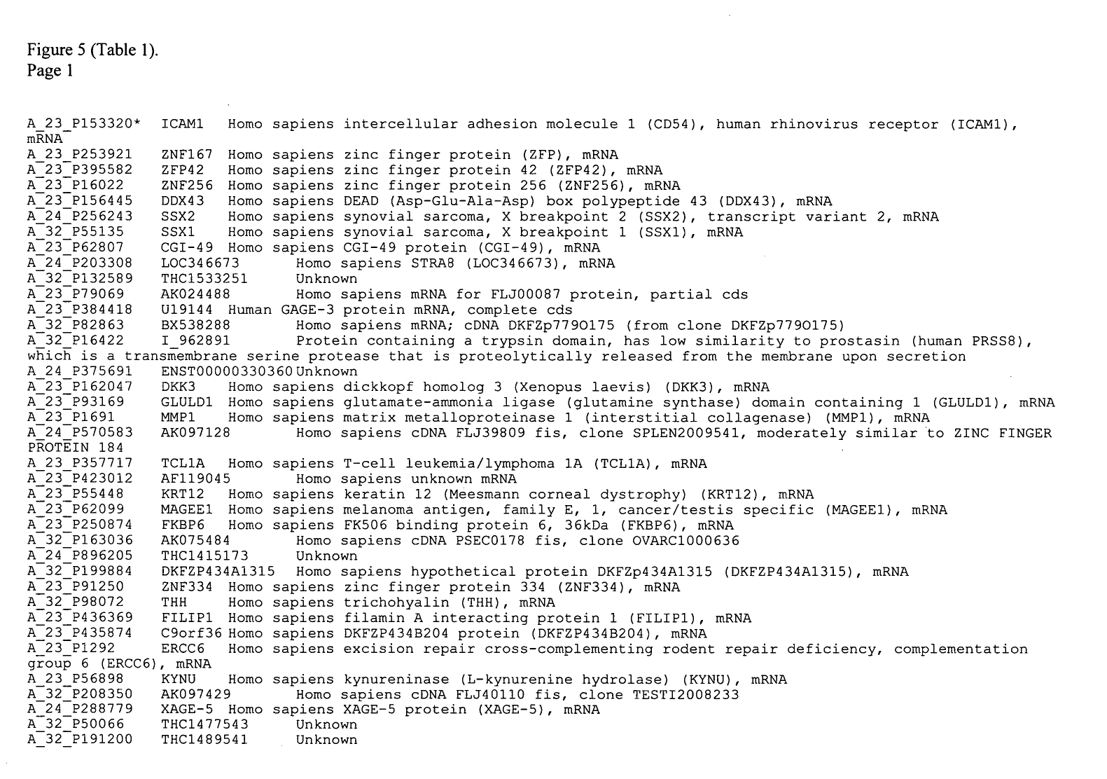

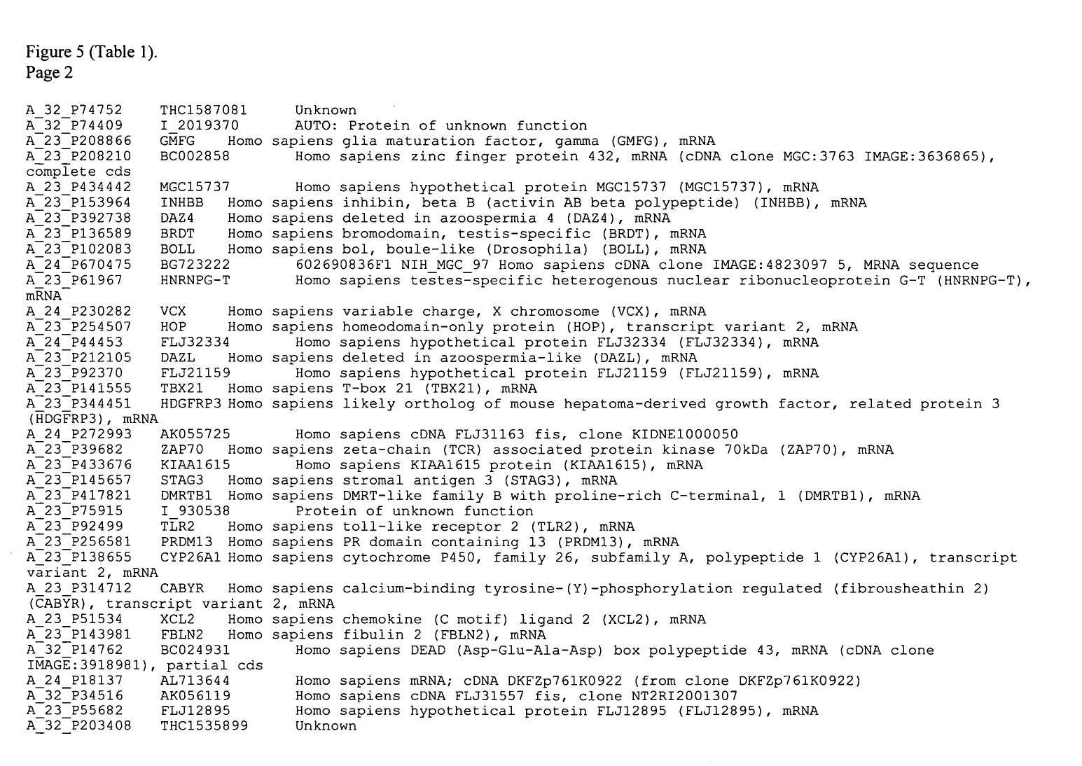

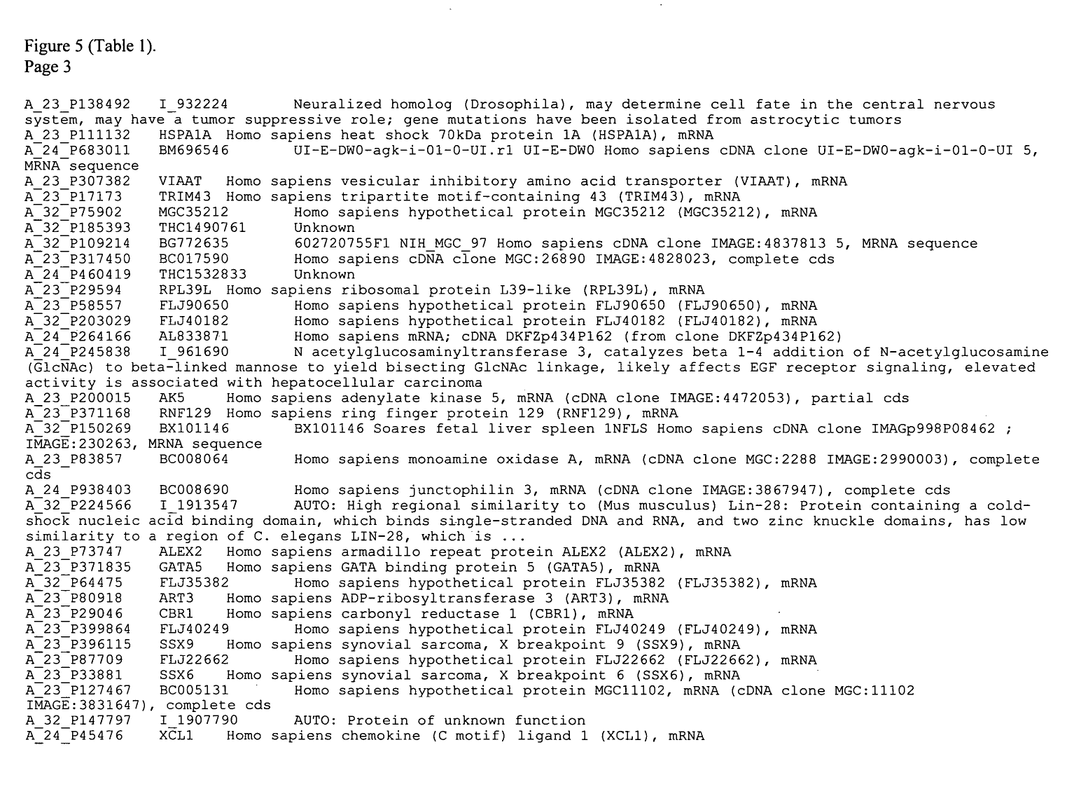

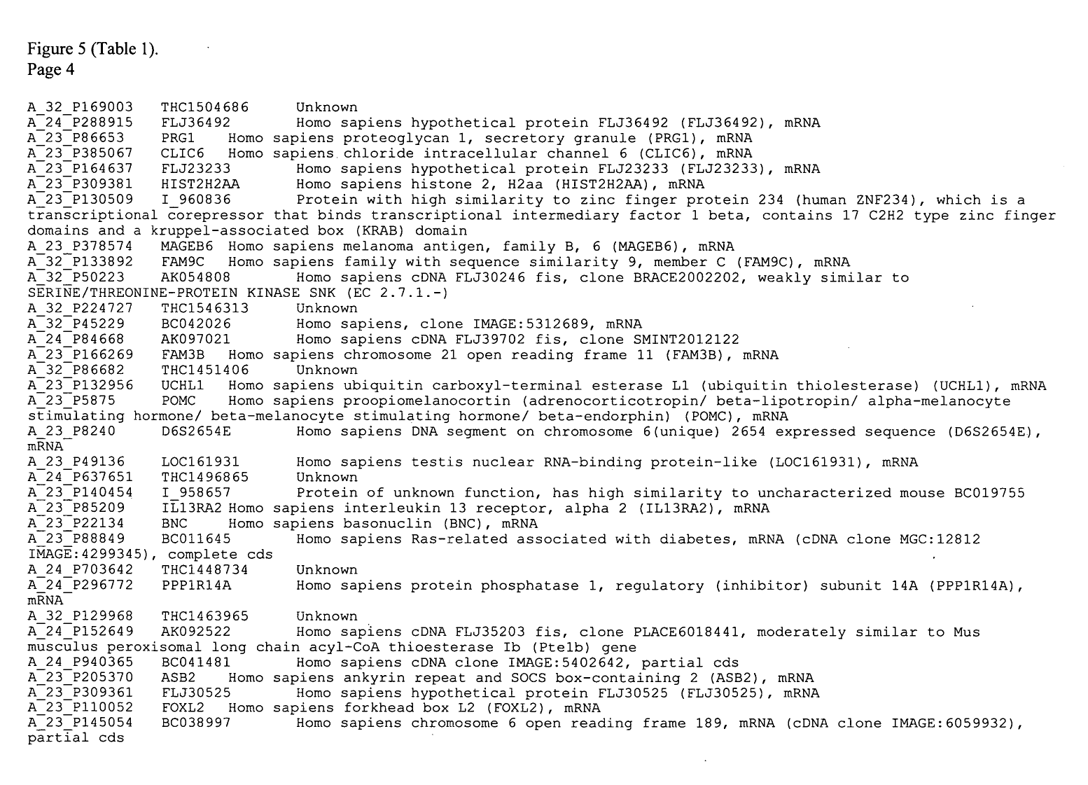

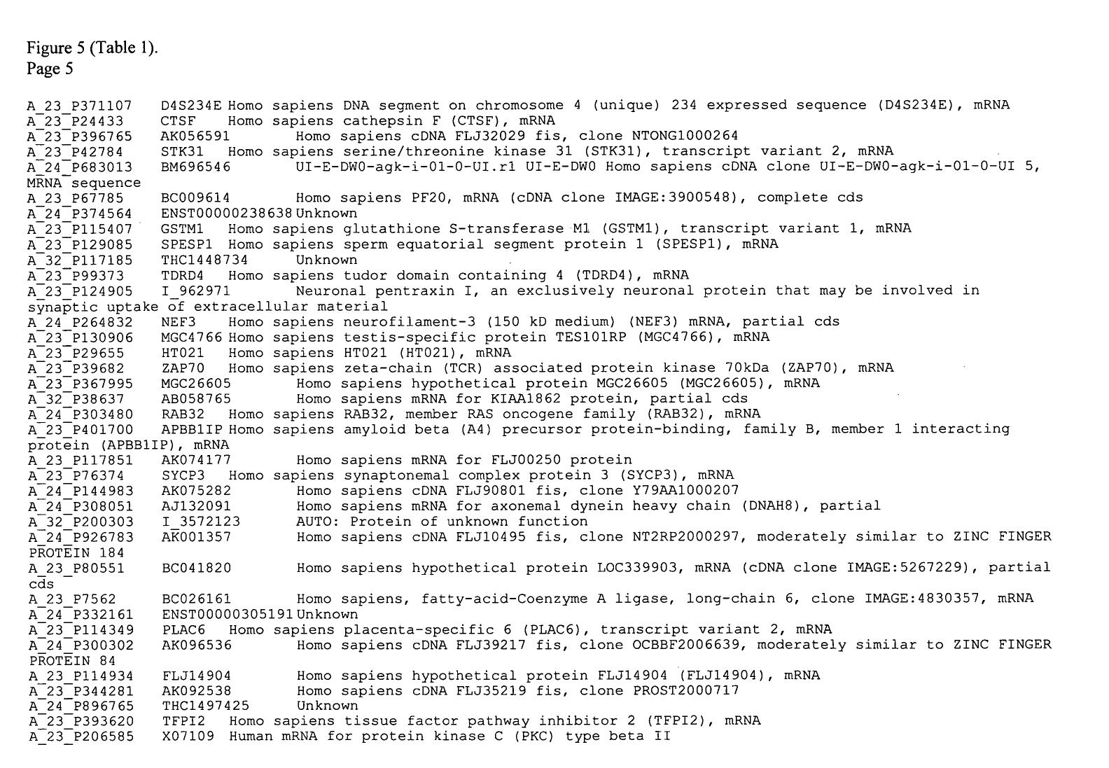

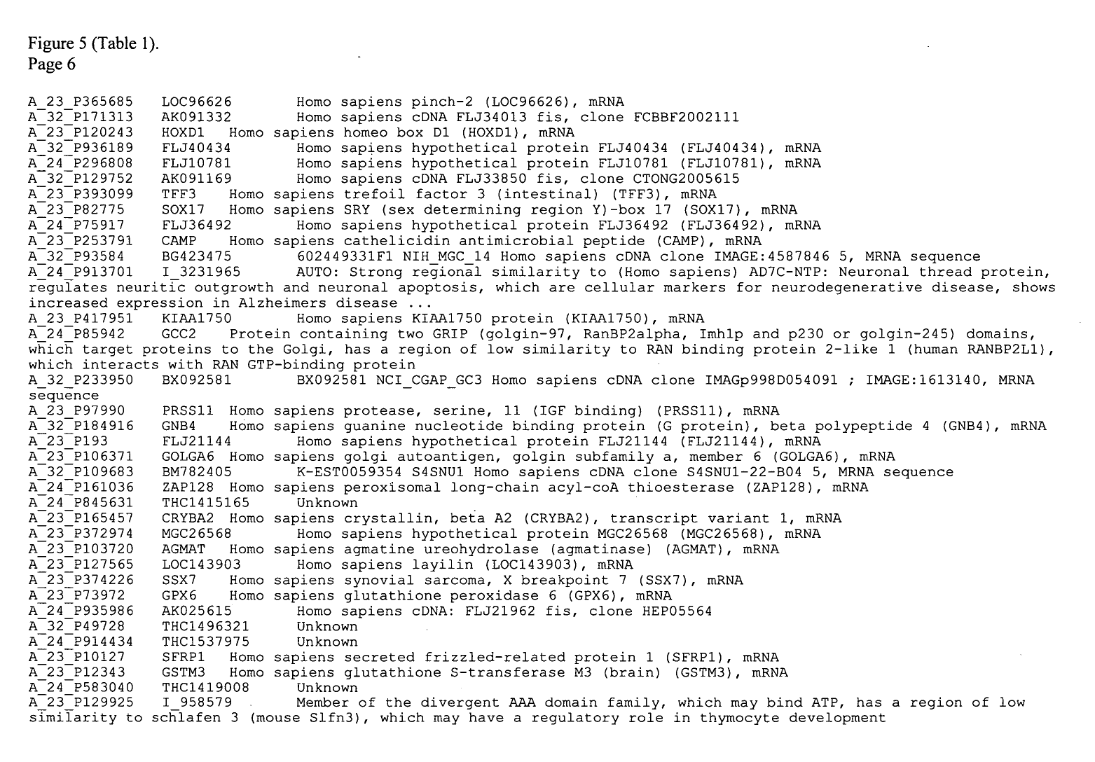

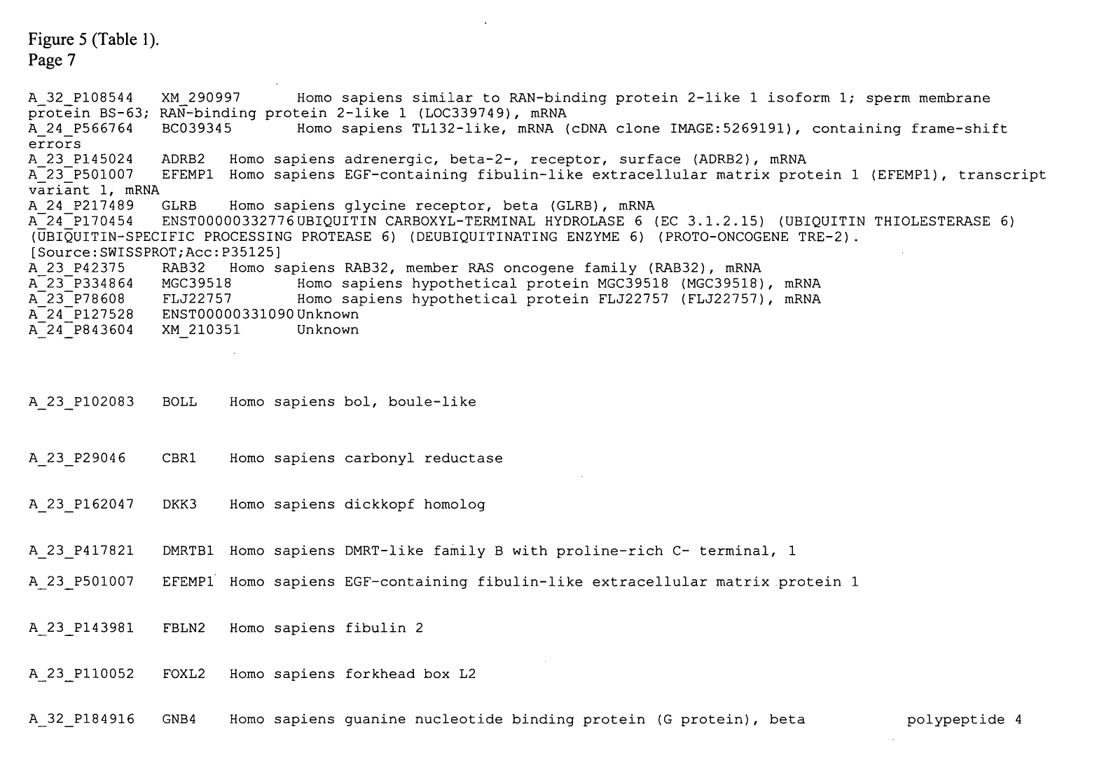

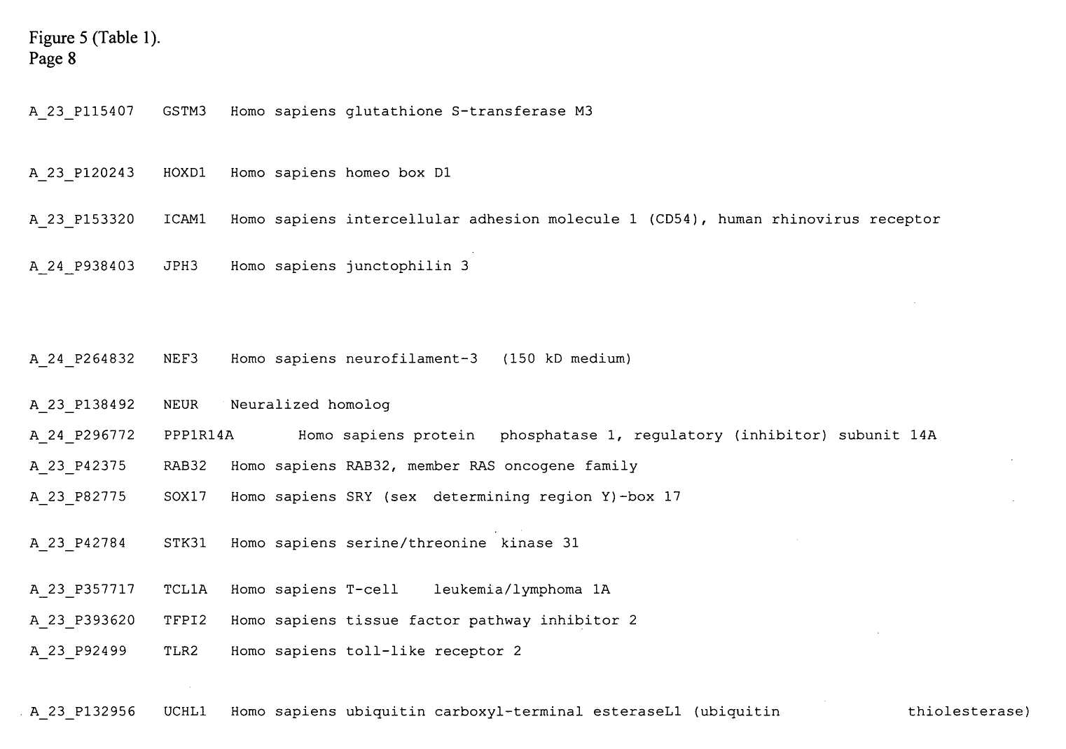

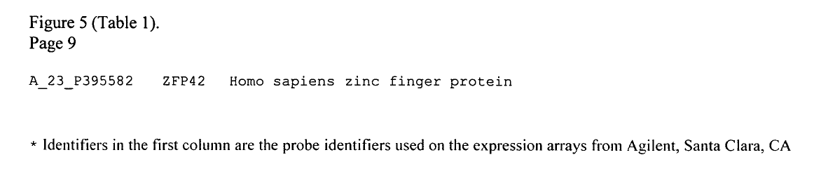

Initial studies confirm that both FOXL2 and Neuralized possess tumor suppressor activity in vitro. When overexpressed in colon cancer cell lines, full-length FOXL2 and Neuralized (FIGS. 4A and C), generate a 10-fold and 20-fold reduction, respectively, in colony growth of HCT116 cells (FIG. 4C), with surviving clones having severely depleted size (FIG. 4B). Similar results were seen in RKO and DLD1 cells (FIG. 4D), both of which have complete gene silencing at the FOXL2 and Neuralized loci. While the precise molecular mechanisms for the growth suppression remains to be determined, Notch signaling has recently been shown to play an important role in differentiation of intestinal crypt cells where deletion of the Notch effector molecule RBP-Jκ or treatment with a highly selective γ-secretase inhibitor was found to be sufficient for conversion of crypt cells to goblet cells (van Es et al.; Fre et al.). Similarly, the closely related FOXL2 transcription factor family member FOXL1 has recently been shown to play a role in epithelial-mesenchymal transition of the intestinal epithelium (Perrault et al.).

In summary, we have devised a microarray gene expression approach with the capacity to define, for any human cancer type for which representative cell culture lines are available, the cancer promoter CpG island DNA “hypermethylome.” Based on the 80% efficiency for identification of the DAC responsive genes in the upper tier of the TSA non-response zone, the HCT116 cells would contain at least ˜200 such genes. We identify these genes in Table 1. Behavior of our guide genes indicates that many more genes also reside in the lower tier of this zone and these could readily be identified from high throughput analysis of the methylation status of the genes in tumor samples. Thus, by identifying the TSA non-responsive zone, a search of only some 2,000 genes out of the whole genome could define the hypermethylome, which appears to constitute hundreds of genes, at least in colon cancer cells, like HCT116, which may harbor the “hypermethylator” phenotype (Toyota et al.²). Definition of the hypermethylome will provide extraordinary information for dissecting the biology of cancer, in terms of identification and functional dissection of key cellular pathways. It will also provide a trove of genes to contribute to the high potential for use of DNA hypermethylation biomarkers in monitoring cancer risk assessment, early diagnosis, and prognosis—and for monitoring the efficacy of targeting reversal of aberrant gene silencing as cancer prevention and/or therapy strategies (Egger et al.).

### Example 3

### Finding New Markers for Early Detection and Prognosis of Colorectal Cancer

Using a high throughput real time methylation specific platform, a total of 240 genomic DNA samples have been analyzed out of which 142 samples were isolated from colorectal cancer and 98 samples haven been isolated from normal colorectal tissue. From each sample, up to 1.5 μg of genomic DNA was converted using a bisulphite based protocol (EZ DNA Methylation Kit™, ZYMO Research, ORANGE, Calif.). After conversion and purification the equivalent of 50 ng of the starting material was applied per sub-array of an OpenArray™ plate on the real-time qPCR system offered by BioTrove Inc. using the DNA double strand specific dye SYBRgreen for signal detection.

The cycling conditions were: 90° C.-10 seconds, (43° C. 18 seconds, 49° C. 60 seconds, 77° C. 22 seconds, 72° C. 70 seconds, 95° C. 28 seconds) for 40 cycles 70° C. for 200 seconds, 45° C. for 5 seconds. A melting curve was created over a temperature range between 45° C. and 94° C. for additional details on product specificity.

Specificity of the primers was tested in independent experiments.

The following primers have been used:

| | | | |
|---|---|---|---|
| gene | ENTREZ | | |
| symbol | Gene ID | Sense primer | Antisense primer |

| | | | |
|---|---|---|---|
| BOLL | 66037 | GTCGTTCGGGGCGAGTATC | CGCCAAACGAACGAAACCG |
| | | (SEQ ID NO: 1) | (SEQ ID NO: 16) |
| CBR1 | 873 | TTAGAGATTAGTTTCGGTTTTCGGTTTGC | CGAAACCTCGCCGAAATACG |
| | | (SEQ ID NO: 2) | (SEQ ID NO: 17) |
| DMRTB1 | 63948 | GCGCGGTTTATTTTAGCGT | ATACGCACCATTTTATCGACC |
| | | (SEQ ID NO: 3) | (SEQ ID NO: 18) |
| EFEMP1 | 2202 | CGGGTTCGTAACGTTGGGTTTAGC | GACAACGACCGCGACG |
| | | (SEQ ID NO: 4) | (SEQ ID NO: 19) |
| FBLN2 | 2199 | TTCGTCGGAGAGGGGGTC | AACGACCTCTAAAAACCGAATCAACG |
| | | (SEQ ID NO: 5) | (SEQ ID NO: 20) |
| FOXL2 | 668 | GCGATAGGTTTTTAGTAAGTAAGCGC | CTCTCCGCTCCAAACGCTAACGCG |
| | | (SEQ ID NO: 6) | (SEQ ID NO: 21) |
| GNB4 | 59345 | GTTGTGAGTTGCGTTTTTTACGTC | CGCTACCGATATCCGCTAAACG |
| | | (SEQ ID NO: 7) | (SEQ ID NO: 22) |
| GSTM3 | 2947 | ATTCGTACGATATGGTGACGGGTTTTC | CGTAAACCCCGCCCCCTTATATCG |
| | | (SEQ ID NO: 8) | (SEQ ID NO: 23) |
| HOXD1 | 3231 | GTCGGTTGACGTTTTGAGATAAGTC | ACCGTCTTCTCGAACGACG |
| | | (SEQ ID NO: 9) | (SEQ ID NO: 24) |
| ICAM1 | 3383 | TAAAGACGTTTTCGCGGTTAAGGTC | ACCACGTCCGAAAAAATCGACG |
| | | (SEQ ID NO: 10) | (SEQ ID NO: 25) |
| NEURL | 9148 | GAGCGTTTAGAACGTTTCGCGTTTC | AAAATCGCTAACGTAAACGTTCGACG |
| | | (SEQ ID NO: 11) | (SEQ ID NO: 26) |
| TCL1A | 8115 | GACGTTATGGTCGAGTGTTCGATATTC | CAAACCCACAAACGATCCGAATAATCG |
| | | (SEQ ID NO: 12) | (SEQ ID NO: 27) |
| TFPI2 | 7980 | GTTCGTTGGGTAAGGCGTTC | CATAAAACGAACACCCGAACCG |
| | | (SEQ ID NO: 13) | (SEQ ID NO: 28) |
| TLR2 | 7097 | GTAGTTATTTGAGAGAACGTCGAGTAGTC | GAACAAACCGACTCGAAAACAACG |
| | | (SEQ ID NO: 14) | (SEQ ID NO: 29) |
| UCHL1 | 7345 | GTTGTATTTTCGCGGAGCGTTC | CTCACAATACGTCTAACCGACG |
| | | (SEQ ID NO: 15) | (SEQ ID NO: 30) |

The primer pairs used amplify the following genomic (NCBI human genome build version 36:2) sequences:

Unconverted Amplicon Sequence

| | |
|---|---|
| BOLL | GCCGTCCGGGGCGAGCACCGGAGCCCGACTGGGCTGAATGGC |
| | AGGTCCTGACCAAGCCACCCGCGCGGCCCCGCCCGCCTGGCG |
| | (SEQ ID NO: 31) |
| CBR1 | CTAGAGACCAGCCTCGGTCTTCGGCCTGCGGGTTCTGCAAAGTCAG |
| | GCTAGCTGGCTCTCCGCCTGCTCCGCACCCCGGCGAGGTTCCG |
| | (SEQ ID NO: 32) |
| DMRTB1 | GCGCGGCTCATCCCAGCGCCACTTGCTCTGCAGCTCCCAGAGGTGGT |
| | GGTTGTGTTACGAAGGCTGACCCTGCCAATGGCCGACAAAATGGTGC |
| | GCAC |
| | (SEQ ID NO: 33) |
| EFEMP1 | CGGGCTCGCAACGCTGGGCTCAGCGCTCGCGCCTCCCTCAGCTCTCT |
| | CCTCCGCCCCCCTTCGCCCTCCCCCTTTCCCTCCCTTTCTCCTCCTCC |
| | TCCTGCCGCCGCGGCCGCTGCC |
| | (SEQ ID NO: 34) |
| FBLN2 | CCCGCCGGAGAGGGGGCCGGGCCGGCGCCGCTCGCTCAGAGCC |
| | CAGACTCGCTGACCCGGCTCCTAGAGGCCGCC |
| | (SEQ ID NO: 35) |
| FOXL2 | GCGACAGGCCTCCAGCAAGCAAGCGCGGGCGGCATCCGCAGTCTC |
| | CAGAAGTTTGAGACTTGGCCGTAAGCGGACTCGTGCGCCCCAACTC |
| | TTTGCCGCGCCAGCGCCTGGAGCGGAGAG |
| | (SEQ ID NO: 36) |
| GNB4 | GCTGTGAGCTGCGCTCTCCACGCCGGCTCCGCGCTCCAGGGGCTG |
| | CTGAGCGCCCAGCGGACACCGGCAGCG |
| | (SEQ ID NO: 37) |
| GSTM3 | ACTCGCACGACATGGTGACGGGCTTCCGAGCCTTCGAGGACTAG |
| | GGAAACTGTGAGCGGGAGGGGCTTTATACCCGACATAAGGGGGCGGGGCCC |
| | ACG |
| | (SEQ ID NO: 38) |
| HOXD1 | GTCGGCTGACGCTTTGAGACAAGCCGGAAAAGGGCCGGGTTCGC |
| | CGAAGGCCGCGTAATCCACCTGGCCGCTGAGGAGGAAAGAGCCGCCGCCCG |
| | AGAAGACGGC |
| | (SEQ ID NO: 39) |
| ICAM1 | TAAAGACGCCTCCGCGGCCAAGGCCGAAAGGGGAAGCGAGGAG |
| | GCCGCCGGGGTGAGTGCCCTCGGGTGTAGAGAGAGGACGCCGA |
| | TTTCCCCGGACGTGGT |
| | (SEQ ID NO: 40) |
| NEURL | GAGCGCCCAGAACGCCCCGCGCTCCGCCGAGCCCCGCTCCAC |
| | GCAGACCCGCGGGCGGGAGGGAGCCACGCACATCGCCGCCGCG |
| | GCCGTCTCCGCGGGGCGGTAACCGAGCCTGCCTCGGAGCCGCCG |
| | AACGCCCACGCCAGCGACCCT |
| | (SEQ ID NO: 41) |
| TCL1A | GACGCCATGGCCGAGTGCCCGACACTCGGGGAGGCAGTCACC |
| | GACCACCCGGACCGCCTGTGGGCCTG |
| | (SEQ ID NO: 42) |
| TFPI2 | GCCCGCTGGGCAAGGCGTCCGAGAAAGCGCCTGGCGGGAG |
| | GAGGTGCGCGGCTTTCTGCTCCAGGCGGCCCGGGTGCCCGCTTTATG |
| | (SEQ ID NO: 43) |
| TLR2 | GCAGTCACCTGAGAGAACGCCGAGCAGCCGCCTGGCTGCGC |
| | TTTCTCGCTGCCTCCGAGCCGGCCTGCCC |
| | (SEQ ID NO: 44) |
| UCHL1 | GCTGCATCTTCGCGGAGCGCCCGGCAGAAATAGCCTAGGGAAGA |
| | CGAAAAACAGCTAGCGGAGCCGCCCAGGCTGCAGCTATAAAGCG |
| | CCGGCCAGACGCACTGTGAG |
| | (SEQ ID NO: 45) |

The following sensitivity (methylation counts per assay in cancer samples/total count of cancer samples tested) and specificity (methylation counts per assay in normal samples/total count of normal samples tested) were determined:

| | Specificity | Sensitivity |
|---|---|---|
| BOLL | 62% | 48% |
| CBR1 | 96% | 14% |
| DMRTB1 | 55% | 39% |
| EFEMP1 | 55% | 31% |
| FBLN2 | 86% | 26% |
| FOXL2 | 83% | 21% |
| GNB4 | 70% | 42% |
| GSTM3 | 92% | 13% |
| HOXD1 | 94% | 16% |
| ICAM1 | 91% | 21% |
| NEURL | 71% | 32% |
| TCL1A | 54% | 51% |
| TFPI2 | 95% | 25% |
| TLR2 | 96% | 10% |
| UCHL1 | 97% | 13% |

### Example 4

Using a real-time PCR based methylation specific PCR platform (Lightcycler™, Roche Applied Sciences), a total of 80 genomic DNA samples have been analyzed out of which 40 samples were isolated from colorectal cancer and 40 samples haven been isolated from normal colorectal tissue. From each sample, up to 1.5 μg of genomic DNA was converted using a bisulphite based protocol (EZ DNA Methylation Kit™, ZYMO Research, ORANGE, Calif.). After conversion and purification the equivalent of 10 ng of the starting material was used per real time PCR reaction using the DNA double strand specific dye SYBRgreen™ for signal detection. The sense primer GTTCGTTGGGTAAGGCGTTC (SEQ ID NO: 46) and the antisense primer CATAAAACGAACACCCGAACCG (SEQ ID NO: 47) were used to perform real-time MSP. The cycling conditions were: activation 95° C.-10 minutes, amplification (95° C. 10 seconds denaturation, 60° C. 30 seconds annealing and extension, 72 C 1 second for measurement) for 45 cycles, melting curve (95° C. for 5 seconds, 45° C. for 1 minute, increase temperature to 95° C., measure every 0.2° C.). Cool down to 45° C.

This experiment led to the following finding:

| Assay | Specificity | Sensitivity |
|---|---|---|
| TFPI | 97% | 69% |

### Example 5

Using a real-time PCR based methylation specific PCR platform (Lightcycler™, Roche Applied Sciences), a total of 90 genomic DNA samples have been analyzed out of which 43 samples were isolated from colorectal cancer and 47 samples haven been isolated from normal colorectal tissue. From each sample, up to 1.5 μg of genomic DNA was converted using a bisulphite based protocol (EZ DNA Methylation Kit™, ZYMO Research, ORANGE, Calif.). After conversion and purification the equivalent of 10 ng of the starting material was used per real time PCR reaction using a probe based detection system. The sense primer GTTCGTTGGGTAAGGCGTTC (SEQ ID NO: 48), the antisense primer CATAAAACGAACACCCGAACCG (SEQ ID NO: 49), and the molecular beacon mCGACATGCACCGCGCACCTCCTCCCGCCAAGCATGTCGv (SEQ ID NO: 50) were used during real-time MSP detection. Cycling conditions were: activation 95° C.-5 minutes, amplification (95° C. 30 seconds denaturation, 57° C. 30 seconds annealing, 72° C. 30 seconds extension and measurement) for 45 cycles. Cool down to 40° C.

This experiment led to the following finding:

| Assay | Specificity | Sensitivity |
|---|---|---|
| TFPI | 85% | 81% |

### Example 6

Using a real-time PCR based methylation specific PCR platform (7900HT fast real-time PCR system, Applied Biosystems), a total of 139 genomic DNA samples have been analyzed out of which 65 samples were isolated from colorectal cancer tissue and 74 samples were isolated from normal colorectal tissue. Real-time MSP: DNA was bisulphite modified using the commercially available EZ DNA Methylation kit from Zymo Research. Analyte quantitations were done in real-time methylation specific PCR assays. The amplicons created during the amplification process were quantified by real-time measurement of the emitted fluorescence.

After conversion and purification the equivalent of 48 ng of the starting material was used per real time PCR reaction using a probe based detection system. The sense primer TTAGATTTCGTAAACGGTGAAAAC (SEQ ID NO: 51), the antisense primer TCTCCTCCGAAAAACGCTC (SEQ ID NO: 52), and the molecular beacon m CGTCTGCAACCGCCGACGACCGCGACGCAGACGv (SEQ ID NO: 53) were used during real-time MSP detection. Cycling conditions were: activation 95° C.-5 minutes, amplification (95° C. 30 seconds denaturation, 57° C. 30 seconds annealing, 72° C. 30 seconds extension and measurement) for 45 cycles.

Based on the JPH3 methylation status, the sensitivity for colon cancer was assessed in terms of Ct value, absolute copy number and as a ratio to beta-actin. This experiment led to the following outcome:

| | | JPH3/Actin × |
|---|---|---|
| | JPH3 copies | 1000 Ratio |
| Cutt off | 207 | 55 |
| Sensitivity | 23.1% | 58.5% |
| specificity | 100.0% | 100.0% |
| Number of | 65 | 65 |
| cases | | |
| Number of | 74 | 74 |
| controls | | |

### REFERENCES

1. Herman J G, Baylin S B. Gene silencing in cancer in association with promoter hypermethylation. N Engl J. Med. 2003 Nov. 20; 349(21):2042-54.
2. Bestor T H. The DNA methyltransferases of mammals. Hum Mol. Genet. 2000 October; 9(16):2395-402.
3. Akiyama Y, Watkins N, Suzuki H, Jair K W, van Engeland M, Esteller M, Sakai H, Ren C Y, Yuasa Y, Herman J G, Baylin S B. GATA-4 and GATA-5 transcription factor genes and potential downstream antitumor target genes are epigenetically silenced in colorectal and gastric cancer. Mol Cell Biol. 2003 December; 23(23):8429-39.
4. Toyota M, Sasaki Y, Satoh A, Ogi K, Kikuchi T, Suzuki H, Mita H, Tanaka N, Itoh F, Issa J P, Jair K W, Schuebel K E, Imai K, Tokino T. Epigenetic inactivation of CHFR in human tumors. Proc Natl Acad Sci USA. 2003 Jun. 24; 100(13):7818-23.
5. Antequera F, Bird A. Number of CpG islands and genes in human and mouse. Proc Natl Acad Sci USA. 1993 Dec. 15; 90(24):11995-9.
6. Suzuki H, Watkins D N, Jair K W, Schuebel K E, Markowitz S D, Chen W D, Pretlow T P, Yang B, Akiyama Y, Van Engeland M, Toyota M, Tokino T, Hinoda Y, Imai K, Herman J G, Baylin S B. Epigenetic inactivation of SFRP genes allows constitutive WNT signaling in colorectal cancer. Nat. Genet. 2004 April; 36(4):417-22.
7. Costello J F, Fruhwald M C, Smiraglia D J, Rush L J, Robertson G P, Gao X, Wright F A, Feramisco J D, Peltomaki P, Lang J C, Schuller D E, Yu L, Bloomfield C D, Caligiuri M A, Yates A, Nishikawa R, Su Huang H, Petrelli N J, Zhang X, O'Dorisio M S, Held W A, Cavenee W K, Plass C. Aberrant CpG-island methylation has non-random and tumor-type-specific patterns. Nat. Genet. 2000 February; 24(2):132-8.
8. Suzuki H, Gabrielson E, Chen W, Anbazhagan R, van Engeland M, Weijenberg M P, Herman J G, Baylin S B. A genomic screen for genes upregulated by demethylation and histone deacetylase inhibition in human colorectal cancer. Nat. Genet. 2002 June; 31(2):141-9.
9. Cameron E E, Bachman K E, Myohanen S, Herman J G, Baylin S B. Synergy of demethylation and histone deacetylase inhibition in the re-expression of genes silenced in cancer. Nat. Genet. 1999 January; 21(1):103-7.
10. Wales M M, Biel M A, el Deiry W, Nelkin B D, Issa J P, Cavenee W K, Kuerbitz S J, Baylin S B. p53 activates expression of HIC-1, a new candidate tumor suppressor gene on 17p13.3. Nat. Med. 1995 June; 1(6):570-7.
11. Weber M, Davies J J, Wittig D, Oakeley E J, Haase M, Lam W L, Schubeler D. Chromosome-wide and promoter-specific analyses identify sites of differential DNA methylation in normal and transformed human cells. Nat. Genet. 2005 August; 37(8):853-62.
12. Hu M, Yao J, Cai L, Bachman K E, van den Brule F, Velculescu V, Polyak K. Distinct epigenetic changes in the stromal cells of breast cancers. Nat. Genet. 2005 August; 37(8):899-905.
13. Gius D, Cui H, Bradbury C M, Cook J, Smart D K, Zhao S, Young L, Brandenburg S A, Hu Y, Bisht K S, Ho A S, Mattson D, Sun L, Munson P J, Chuang E Y, Mitchell J B, Feinberg A P. Distinct effects on gene expression of chemical and genetic manipulation of the cancer epigenome revealed by a multimodality approach. Cancer Cell. 2004 October; 6(4):361-71.
14. Paz M F, Wei S, Cigudosa J C, Rodriguez-Perales S, Peinado M A, Huang T H, Esteller M. Genetic unmasking of epigenetically silenced tumor suppressor genes in colon cancer cells deficient in DNA methyltransferases. Hum Mol. Genet. 2003 Sep. 1; 12(17):2209-19.
15. Toyota M, Ho C, Ahuja N, Jair K W, Li Q, Ohe-Toyota M, Baylin S B, Issa J P. Identification of differentially methylated sequences in colorectal cancer by methylated CpG island amplification. Cancer Res. 1999 May 15; 59(10):2307-12.
16. Keshet I, Schlesinger Y, Farkash S, Rand E, Hecht M, Segal E, Pikarski E, Young R A, Niveleau A, Cedar H, Simon I. Evidence for an instructive mechanism of de novo methylation in cancer cells. Nat. Genet. 2006 February; 38(2):149-53.
17. Rhee I, Bachman K E, Park B H, Jair K W, Yen R W, Schuebel K E, Cui H, Feinberg A P, Lengauer C, Kinzler K W, Baylin S B, Vogelstein B. DNMT1 and DNMT3b cooperate to silence genes in human cancer cells. Nature. 2002 Apr. 4; 416(6880):552-6.
18. Rhee I, Jair K W, Yen R W, Lengauer C, Herman J G, Kinzler K W, Vogelstein B, Baylin S B, Schuebel K E. CpG methylation is maintained in human cancer cells lacking DNMT1. Nature. 2000 Apr. 27; 404(6781): 1003-7.
19. Herman J G, Graff J R, Myohanen S, Nelkin B D, Baylin S B. Methylation-specific PCR: a novel PCR assay for methylation status of CpG islands. Proc Natl Acad Sci USA. 1996 Sep. 3; 93(18):9821-6.
20. Herman J G, Umar A, Polyak K, Graff J R, Ahuja N, Issa J P, Markowitz S, Willson J K, Hamilton S R, Kinzler K W, Kane M F, Kolodner R D, Vogelstein B, Kunkel T A, Baylin S B. Incidence and functional consequences of hMLH1 promoter hypermethylation in colorectal carcinoma. Proc Natl Acad Sci USA. 1998 Jun. 9; 95(12):6870-5.
21. van Es J H, van Gijn M E, Riccio 0, van den Born M, Vooijs M, Begthel H, Cozijnsen M, Robine S, Winton D J, Radtke F, Clevers H. Notch/gamma-secretase inhibition turns proliferative cells in intestinal crypts and adenomas into goblet cells. Nature. 2005 Jun. 16; 435(7044):959-63.
22. Fre S, Huyghe M, Mourikis P, Robine S, Louvard D, Artavanis-Tsakonas S, Notch signals control the fate of immature progenitor cells in the intestine. Nature. 2005 Jun. 16; 435(7044):964-8.
23. Perreault N, Sackett S D, Katz J P, Furth E E, Kaestner K H. Fox11 is a mesenchymal Modifier of Min in carcinogenesis of stomach and colon. Genes Dev. 2005 Feb. 1; 19(3):311-5.
24. Crisponi L, Deiana M, Loi A, Chiappe F, Uda M, Amati P, Bisceglia L, Zelante L, Nagaraja R, Porcu S, Ristaldi M S, Marzella R, Rocchi M, Nicolino M, Lienhardt-Roussie A, Nivelon A, Verloes A, Schlessinger D, Gasparini P, Bonneau D, Cao A, Pilia G. The putative forkhead transcription factor FOXL2 is mutated in blepharophimosis/ptosis/epicanthus inversus syndrome. Nat. Genet. 2001 February; 27(2):159-66.
25. Lehmann O J, Sowden J C, Carlsson P, Jordan T, Bhattacharya S S. Fox's in development and disease. Trends Genet. 2003 June; 19(6):339-44.
26. Nakamura, H.; Yoshida, M.; Tsuiki, H.; Ito, K.; Ueno, M.; Nakao, M.; Oka, K.; Tada, M.; Kochi, M.; Kuratsu, J.; Ushio, Y.; Saya, H.: Identification of a human homolog of the Drosophila neuralized gene within the 10q25.1 malignant astrocytoma deletion region. Oncogene 16: 1009-1019, 1998.
27. Pavlopoulos E, Pitsouli C, Klueg K M, Muskavitch M A, Moschonas N K, Delidakis C. neuralized Encodes a peripheral membrane protein involved in delta signaling and endocytosis. Dev Cell. 2001 December; 1(6):807-16.
28. Deblandre G A, Lai E C, Kintner C. Xenopus neuralized is a ubiquitin ligase that interacts with XDelta1 and regulates Notch signaling. Dev Cell. 2001 December; 1(6):795-806.
29. Lai E C, Deblandre G A, Kintner C, Rubin G M. Drosophila neuralized is a ubiquitin ligase that promotes the internalization and degradation of delta. Dev Cell. 2001 December; 1 (6):783-94.
30. Ting A H, Jair K W, Suzuki H, Yen R W, Baylin S B, Schuebel K E. CpG island hypermethylation is maintained in human colorectal cancer cells after RNAi-mediated depletion of DNMT1. Nat. Genet. 2004 June; 36(6):582-4.
31. Ushijima T. Detection and interpretation of altered methylation patterns in cancer cells. Nat Rev Cancer. 2005 March; 5(3):223-31.
32. Bachman K E, Herman J G, Corn P G, Merlo A, Costello J F, Cavenee W K, Baylin S B, Graff J R. Methylation-associated silencing of the tissue inhibitor of metalloproteinase-3 gene suggest a suppressor role in kidney, brain, and other human cancers. Cancer Res. 1999 Feb. 15; 59(4):798-802.
33. Parsons R, Li G M, Longley M J, Fang W H, Papadopoulos N, Jen J, de la Chapelle A, Kinzier K W, Vogelstein B, Modrich P. Hypermutability and mismatch repair deficiency in RER+ tumor cells. Cell. 1993 Dec. 17; 75(6):1227-36.
34. Ionov Y, Peinado M A, Malkhosyan S, Shibata D, Perucho M. Ubiquitous somatic mutations in simple repeated sequences reveal a new mechanism for colonic carcinogenesis. Nature. 1993 Jun. 10; 363(6429):558-61.
35. Leach F S, Nicolaides N C, Papadopoulos N, Liu B, Jen J, Parsons R, Peltomaki P, Sistonen P, Aaltonen L A, Nystrom-Lahti M, et al. Mutations of a mutS homolog in hereditary nonpolyposis colorectal cancer. Cell. 1993 Dec. 17; 75(6):1215-25.
36. Toyota M, Ahuja N, Ohe-Toyota M, Herman J G, Baylin S B, Issa J P. CpG island methylator phenotype in colorectal cancer. Proc Natl Acad Sci USA. 1999 Jul. 20; 96(15):8681-6.
37. Uda M, Ottolenghi C, Crisponi L, Garcia J E, Deiana M, Kimber W, Forabosco A, Cao A, Schlessinger D, Pilia G. Fox12 disruption causes mouse ovarian failure by pervasive blockage of follicle development. Hum Mol. Genet. 2004 Jun. 1; 13(11):1171-81.
38. Ihaka R and Gentleman R. R: A language for data analysis and graphics. Journal of Computational and Graphical Statistics 1996; 5:299-314.
39. Gentleman R C, Carey V J, Bates D M, Bolstad B, Dettling M, Dudoit S, Ellis B, Gautier L, Ge Y, Gentry J, Homik K, Hothom T, Huber W, Iacus S, Irizarry R, Leisch F, Li C, Maechler M, Rossini A J, Sawitzki G, Smith C, Smyth G, Tierney L, Yang J Y, Zhang J. Bioconductor: Open software development for computational biology and bioinformatics. Genome Biology 2004; 5:R80.
40. Smyth, G. K., and Speed, T. P. (2003). Normalization of cDNA microarray data. In: METHODS: Selecting Candidate Genes from DNA Array Screens: Application to Neuroscience, D. Carter (ed.). Methods Volume 31, Issue 4, December 2003, pages 265-273. 11.
41. Smyth, G. K., Yang, Y.-H., Speed, T. P. (2003). Statistical issues in microarray data analysis. In: Functional Genomics: Methods and Protocols, M. J. Brownstein and A. B. Khodursky (eds.), Methods in Molecular Biology Volume 224, Humana Press, Totowa, N.J., pages 111-136.
42. Radtke F, Clevers H. Self-renewal and cancer of the gut: two sides of a coin. Science. 2005 Mar. 25; 307(5717):1904-9.
43. Egger G, Liang G, Aparicio A, Jones P A. Epigenetics in human disease and prospects for epigenetic therapy Nature. 2004 May 27; 429(6990):457-63.

We claim:

1. A method for identifying colorectal cancer or its precursor, or predisposition to colorectal cancer, comprising:
detecting in a test sample containing colorectal cells or nucleic acids from colorectal cells, epigenetic silencing of at least one gene selected from A_{—}23_P132956 UCHL1 Homo sapiens ubiquitin carboxyl-terminal esteraseL1 (ubiquitin thiolesterase); A_{—}23_P29046 CBR1 Homo sapiens carbonyl reductase; A_{—}23_P92499 TLR2 Homo sapiens toll-like receptor 2; A_{—}23_P393620 TFPI2 Homo sapiens tissue factor pathway inhibitor 2; A_{—}23_P120243 HOXD1 Homo sapiens homeo box D1; A_{—}23_P115407 GSTM3 Homo sapiens glutathione S-transferase M3; A_{—}23_P153320 ICAM1 Homo sapiens intercellular adhesion molecule 1 (CD54), human rhinovirus receptor; A_{—}23_P143981 FBLN2 Homo sapiens fibulin 2; A_{—}23_P110052 FOXL2 Homo sapiens forkhead box L2; A_{—}23_P138492 NEURNeuralized homolog; A_{—}32_P184916 GNB4 Homo sapiens guanine nucleotide binding protein (G protein), beta polypeptide 4; and A_{—}24_P938403 JPH3 Homo sapiens junctophilin 3;
identifying the test sample as containing cells that are neoplastic, precursor to neoplastic, or predisposed to neoplasia, or as containing nucleic acids from cells that are neoplastic, precursor to neoplastic, or predisposed to neoplasia.

2. The method of claim 1 wherein the test sample contains adenoma cells or nucleic acids from adenoma cells.

3. The method of claim 1 wherein the test sample contains carcinoma cells or nucleic acids from carcinoma cells.

4. The method of claim 1 wherein the at least one gene is A_{—}23_P132956 UCHL1 Homo sapiens ubiquitin carboxyl-terminal esteraseL1 (ubiquitin thiolesterase).

5. The method of claim 1 wherein the test sample is from a tissue specimen.

6. The method of claim 1 wherein the test sample is from a biopsy specimen.

7. The method of claim 1 wherein the test sample is from a surgical specimen.

8. The method of claim 1 wherein the test sample is from a cytological specimen.

9. The method of claim 1 wherein the test sample is isolated from mucus, stool, blood, serum, or urine.

10. The method of claim 6 wherein surgical removal of neoplastic tissue is recommended to the patient.

11. The method of claim 6 wherein adjuvant chemotherapy is recommended to the patient.

12. The method of claim 6 wherein adjuvant radiation therapy is recommended to the patient.

13. The method of claim 9 wherein a colonoscopy or sigmoidoscopy is recommended to the patient.

14. The method of claim 6 wherein increased frequency of colonoscopy is recommended to the patient.

15. The method of claim 9 wherein an imaging study of the colon is recommended to the patient.

16. The method of claim 1 wherein epigenetic silencing of at least two genes is detected.

17. The method of claim 1 wherein epigenetic silencing is detected by detecting methylation of a CpG dinucleotide motif in the gene.

18. The method of claim 1 wherein epigenetic silencing is detected by detecting methylation of a CpG dinucleotide motif in a promoter of the gene.

19. The method of claim 1 wherein epigenetic silencing is detected by detecting diminished expression of mRNA of the gene.

20. The method of claim 17 wherein methylation is detected by contacting at least a portion of the gene with a methylation-sensitive restriction endonuclease, said endonuclease preferentially cleaving methylated recognition sites relative to non-methylated recognition sites, whereby cleavage of the portion of the gene indicates methylation of the portion of the gene.

21. The method of claim 17 wherein methylation is detected by contacting at least a portion of the gene with a methylation-sensitive restriction endonuclease, said endonuclease preferentially cleaving non-methylated recognition sites relative to methylated recognition sites, whereby cleavage of the portion of the gene indicates non-methylation of the portion of the gene provided that the gene comprises a recognition site for the methylation-sensitive restriction endonuclease.

22. The method of claim 17 wherein methylation is detected by:
contacting at least a portion of the gene of the test cell with a chemical reagent that selectively modifies a non-methylated cytosine residue relative to a methylated cytosine residue, or selectively modifies a methylated cytosine residue relative to a non-methylated cytosine residue; and
detecting a product generated due to said contacting.

23. The method of claim 22 wherein the step of detecting comprises amplification with at least one primer that hybridizes to a sequence comprising a modified non-methylated CpG dinucleotide motif but not to a sequence comprising an unmodified methylated CpG dinucleotide motif thereby forming amplification products.

24. The method of claim 22 wherein the step of detecting comprises amplification with at least one primer that hybridizes to a sequence comprising an unmodified methylated CpG dinucleotide motif but not to a sequence comprising a modified non-methylated CpG dinucleotide motif thereby forming amplification products.

25. The method of claim 22 wherein the product is detected by a method selected from the group consisting of electrophoresis, hybridization, amplification, sequencing, ligase chain reaction, chromatography, mass spectrometry, and combinations thereof.

26. The method of claim 22 wherein the chemical reagent is hydrazine.

27. The method of claim 26 further comprising cleavage of the hydrazine-contacted at least a portion of the gene with piperidine.

28. The method of claim 22 wherein the chemical reagent comprises bisulfite ions.

29. The method of claim 28 further comprising treating the bisulfite ion-contacted at least a portion of the gene with alkali.

30. A method of reducing or inhibiting neoplastic growth of a cell which exhibits epigenetic silenced transcription of at least one gene associated with a cancer, the method comprising:
determining that a cell has an epigenetic silenced gene selected from A_{—}23_P132956 UCHL1 Homo sapiens ubiquitin carboxyl-terminal esteraseL1 (ubiquitin thiolesterase); A_{—}23_P29046 CBR1 Homo sapiens carbonyl reductase; A_{—}23_P92499 TLR2 Homo sapiens toll-like receptor 2; A_{—}23_P393620 TFPI2 Homo sapiens tissue factor pathway inhibitor 2; A_{—}23_P120243 HOXD1 Homo sapiens homeo box D1; A_{—}23_P115407 GSTM3 Homo sapiens glutathione S-transferase M3; A_{—}23_P153320 ICAM1 Homo sapiens intercellular adhesion molecule 1 (CD54), human rhinovirus receptor; A_{—}23_P143981 FBLN2 Homo sapiens fibulin 2; A_{—}23_P110052 FOXL2 Homo sapiens forkhead box L2; A_{—}23_P 138492 NEURNeuralized homolog; A_{—}32_P 184916 GNB4 Homo sapiens guanine nucleotide binding protein (G protein), beta polypeptide 4; and A_{—}24_P938403 JPH3 Homo sapiens junctophilin 3;
restoring expression of a polypeptide encoded by the epigenetic silenced gene in the cell by contacting the cell with a CpG dinucleotide demethylating agent, thereby reducing or inhibiting unregulated growth of the cell.

31. The method of claim 30 wherein the gene is A_{—}23_P132956 UCHL1 Homo sapiens ubiquitin carboxyl-terminal esteraseL1 (ubiquitin thiolesterase).

32. The method of claim 30 wherein the contacting is performed in vitro.

33. The method of claim 30 wherein the contacting is performed in vivo by administering the agent to a mammalian subject comprising the cell.

34. The method of claim 30 wherein the demethylating agent is selected from the group consisting of 5-aza-2′-deoxycytidine, 5-aza-cytidine, Zebularine, procaine, and L-ethionine.

35. A method of reducing or inhibiting neoplastic growth of a cell which exhibits epigenetic silenced transcription of at least one gene associated with a cancer, the method comprising:
determining that a cell has an epigenetic silenced gene selected from A_{—}23_P132956 UCHL1 Homo sapiens ubiquitin carboxyl-terminal esteraseL1 (ubiquitin thiolesterase); A_{—}23_P29046 CBR1 Homo sapiens carbonyl reductase; A_{—}23_P92499 TLR2 Homo sapiens toll-like receptor 2; A_{—}23_P393620 TFPI2 Homo sapiens tissue factor pathway inhibitor 2; A_{—}23_P120243 HOXD1 Homo sapiens homeo box D1; A_{—}23_P115407 GSTM3 Homo sapiens glutathione S-transferase M3; A_{—}23_P153320 ICAM1 Homo sapiens intercellular adhesion molecule 1 (CD54), human rhinovirus receptor; A_{—}23_P143981 FBLN2 Homo sapiens fibulin 2; A_{—}23_P110052 FOXL2 Homo sapiens forkhead box L2; A_{—}23_P138492 NEUR Neuralized homolog; A_{—}32_P184916 GNB4 Homo sapiens guanine nucleotide binding protein (G protein), beta polypeptide 4; and A_{—}24_P938403 JPH3 Homo sapiens junctophilin 3;
introducing a polynucleotide encoding a polypeptide into the cell, wherein the polypeptide is encoded by said gene, wherein the polypeptide is expressed in the cell thereby restoring expression of the polypeptide in the cell.

36. The method of claim 35 wherein the gene is A_{—}23_P132956 UCHL1 Homo sapiens ubiquitin carboxyl-terminal esteraseL1 (ubiquitin thiolesterase).

37. A method of treating a cancer patient, the method comprising:
determining that a cancer cell in the patient has an epigenetic silenced gene selected from A_{—}23_P132956 UCHL1 Homo sapiens ubiquitin carboxyl-terminal esteraseL1 (ubiquitin thiolesterase); A_{—}23_P29046 CBR1 Homo sapiens carbonyl reductase; A_{—}23_P92499 TLR2 Homo sapiens toll-like receptor 2; A_{—}23_P393620 TFPI2 Homo sapiens tissue factor pathway inhibitor 2; A_{—}23_P120243 HOXD1 Homo sapiens homeo box D1; A_{—}23_P115407 GSTM3 Homo sapiens glutathione S-transferase M3; A_{—}23_P153320 ICAM1 Homo sapiens intercellular adhesion molecule 1 (CD54), human rhinovirus receptor; A_{—}23_P143981 FBLN2 Homo sapiens fibulin 2; A_{—}23_P110052 FOXL2 Homo sapiens forkhead box L2; A_{—}23_P138492 NEUR Neuralized homolog; A_{—}32_P184916 GNB4 Homo sapiens guanine nucleotide binding protein (G protein), beta polypeptide 4; and A_{—}24_P938403 JPH3 Homo sapiens junctophilin 3;
administering a demethylating agent to the patient in sufficient amounts to restore expression of the epigenetic silenced gene in the patient's cancer cells.

38. The method of claim 37 wherein the demethylating agent is selected from the group consisting of 5-aza-2′-deoxycytidine, 5-aza-cytidine, Zebularine, procaine, and L-ethionine.

39. The method of claim 37 wherein the gene is A_{—}23_P132956 UCHL1 Homo sapiens ubiquitin carboxyl-terminal esteraseL1 (ubiquitin thiolesterase).

40. A method of treating a cancer patient, the method comprising:
determining that a cancer cell in the patient has an epigenetic silenced gene selected from A_{—}23_P132956 UCHL1 Homo sapiens ubiquitin carboxyl-terminal esteraseL1 (ubiquitin thiolesterase); A_{—}23_P29046 CBR1 Homo sapiens carbonyl reductase; A_{—}23_P92499 TLR2 Homo sapiens toll-like receptor 2; A_{—}23_P393620 TFPI2 Homo sapiens tissue factor pathway inhibitor 2; A_{—}23_P120243 HOXD1 Homo sapiens homeo box D1; A_{—}23_P115407 GSTM3 Homo sapiens glutathione S-transferase M3; A_{—}23_P153320 ICAM1 Homo sapiens intercellular adhesion molecule 1 (CD54), human rhinovirus receptor; A 23_P143981 FBLN2 Homo sapiens fibulin 2; A_{—}23_P110052 FOXL2 Homo sapiens forkhead box L2; A_{—}23_P138492 NEUR Neuralized homolog; A_{—}32_P184916 GNB4 Homo sapiens guanine nucleotide binding protein (G protein), beta polypeptide 4; and A_{—}24_P938403 JPH3 Homo sapiens junctophilin 3;
administering to the patient a polynucleotide encoding a polypeptide, wherein the polypeptide is encoded by the epigenetic silenced gene, wherein the polypeptide is expressed in the patient's tumor thereby restoring expression of the polypeptide in the cancer.

41. The method of claim 40 wherein the epigenetic silenced gene is A_{—}23_P132956 UCHL1 Homo sapiens ubiquitin carboxyl-terminal esteraseL1 (ubiquitin thiolesterase).

42. A method for selecting a therapeutic strategy for treating a cancer patient, comprising:
identifying a gene whose expression in cancer cells of the patient is reactivated by a demethylating agent, wherein the gene is selected from A_{—}23_P132956 UCHL1 Homo sapiens ubiquitin carboxyl-terminal esteraseL1 (ubiquitin thiolesterase); A_{—}23_P29046 CBR1 Homo sapiens carbonyl reductase; A_{—}23_P92499 TLR2 Homo sapiens toll-like receptor 2; A_{—}23_P393620 TFPI2 Homo sapiens tissue factor pathway inhibitor 2; A_{—}23_P120243 HOXD1 Homo sapiens homeo box D1; A_{—}23_P115407 GSTM3 Homo sapiens glutathione S-transferase M3; A_{—}23_P153320 ICAM1 Homo sapiens intercellular adhesion molecule 1 (CD54), human rhinovirus receptor; A_{—}23_P143981 FBLN2 Homo sapiens fibulin 2; A_{—}23_P110052 FOXL2 Homo sapiens forkhead box L2; A_{—}23_P138492 NEUR Neuralized homolog; A_{—}32_P184916 GNB4 Homo sapiens guanine nucleotide binding protein (G protein), beta polypeptide 4; and A_{—}24_P938403 JPH3 Homo sapiens junctophilin 3; and
selecting a therapeutic agent which increases expression of the gene for treating said cancer patient.

43. The method of claim 42 wherein the gene is A_{—}23_P132956 UCHL1 Homo sapiens ubiquitin carboxyl-terminal esteraseL1 (ubiquitin thiolesterase).

44. The method of claim 42 further comprising the step of prescribing the therapeutic agent for said cancer patient.

45. The method of claim 42 further comprising the step of administering the therapeutic agent to said cancer patient.

46. The method of claim 42 wherein the therapeutic agent comprises a polynucleotide encoding the gene.

47. The method of claim 42 wherein the demethylating agent is 5-aza-2′-deoxycytidine.

48. The method of claim 42 wherein the therapeutic agent is 5-aza-2′-deoxycytidine.

49. The method of claim 42 wherein the cancer cells are obtained from a surgical specimen.

50. The method of claim 42 wherein the cancer cells are obtained from a biopsy specimen.

51. The method of claim 42 wherein the cancer cells are obtained from a cytological sample.

52. The method of claim 42 wherein the cancer cells are obtained from stool, mucus, serum, blood, or urine.

53. A kit for assessing methylation in a test sample, comprising in a package:
a reagent that (a) modifies methylated cytosine residues but not non-methylated cytosine residues, or that (b) modifies non-methylated cytosine residues but not methylated cytosine residues; and
a pair of oligonucleotide primers that specifically hybridizes under amplification conditions to a region of a gene selected from A_{—}23_P132956 UCHL1 Homo sapiens ubiquitin carboxyl-terminal esteraseL1 (ubiquitin thiolesterase); A_{—}23_P29046 CBR1 Homo sapiens carbonyl reductase; A_{—}23_P92499 TLR2 Homo sapiens toll-like receptor 2; A_{—}23_P393620 TFPI2 Homo sapiens tissue factor pathway inhibitor 2; A_{—}23_P120243 HOXD1 Homo sapiens homeo box D1; A_{—}23_P115407 GSTM3 Homo sapiens glutathione S-transferase M3; A_{—}23_P153320 ICAM1 Homo sapiens intercellular adhesion molecule 1 (CD54), human rhinovirus receptor; A_{—}23_P143981 FBLN2 Homo sapiens fibulin 2; A_{—}23_P110052 FOXL2 Homo sapiens forkhead box L2; A_{—}23_P138492 NEUR Neuralized homolog; A_{—}32_P184916 GNB4 Homo sapiens guanine nucleotide binding protein (G protein), beta polypeptide 4; and A_{—}24_P938403 JPH3 Homo sapiens junctophilin 3; wherein the region is within about 1 kb of said gene's transcription start site.

54. The kit of claim 53 wherein the gene is A_{—}23_P132956 UCHL1 Homo sapiens ubiquitin carboxyl-terminal esteraseL1 (ubiquitin thiolesterase).

55. The kit of claim 53 wherein at least one of said pair of oligonucleotide primers hybridizes to a sequence comprising a modified non-methylated CpG dinucleotide motif but not to a sequence comprising an unmodified methylated CpG dinucleotide motif or wherein at least one of said pair of oligonucleotide primers hybridizes to a sequence comprising an unmodified methylated CpG dinucleotide motif but not to sequence comprising a modified non-methylated CpG dinucleotide motif.

56. The kit of claim 55 further comprising (a) a first oligonucleotide probe which hybridizes to a sequence comprising a modified non-methylated CpG dinucleotide motif but not to a sequence comprising an unmodified methylated CpG dinucleotide motif, (b) a second oligonucleotide probe that hybridizes to a sequence comprising an unmodified methylated CpG dinucleotide motif but not to sequence comprising a modified non-methylated CpG dinucleotide motif, or (c) both said first and second oligonucleotide probes.

57. The kit of claim 53 further comprising (a) a first oligonucleotide probe which hybridizes to a sequence comprising a modified non-methylated CpG dinucleotide motif but not to a sequence comprising an unmodified methylated CpG dinucleotide motif, (b) a second oligonucleotide probe that hybridizes to a sequence comprising an unmodified methylated CpG dinucleotide motif but not to sequence comprising a modified non-methylated CpG dinucleotide motif, or (c) both said first and second oligonucleotide probes.

58. The kit of claim 53 further comprising an oligonucleotide probe.

59. The kit of claim 53 further comprising a DNA polymerase for amplifying DNA.


SEQUENCE LISTING (52 sequences)

SEQ ID NO: 1 - DNA, 19 bp, Homo sapiens
gtcgttcggggcgagtatc

SEQ ID NO: 2 - DNA, 29 bp, Homo sapiens
ttagagattagtttcggttttcggtttgc

SEQ ID NO: 3 - DNA, 19 bp, Homo sapiens
gcgcggtttattttagcgt

SEQ ID NO: 4 - DNA, 24 bp, Homo sapiens
cgggttcgtaacgttgggtttagc

SEQ ID NO: 5 - DNA, 18 bp, Homo sapiens
ttcgtcggagagggggtc

SEQ ID NO: 6 - DNA, 26 bp, Homo sapiens
gcgataggtttttagtaagtaagcgc

SEQ ID NO: 7 - DNA, 24 bp, Homo sapiens
gttgtgagttgcgttttttacgtc

SEQ ID NO: 8 - DNA, 27 bp, Homo sapiens
attcgtacgatatggtgacgggttttc

SEQ ID NO: 9 - DNA, 25 bp, Homo sapiens
gtcggttgacgttttgagataagtc

SEQ ID NO: 10 - DNA, 25 bp, Homo sapiens
taaagacgttttcgcggttaaggtc

SEQ ID NO: 11 - DNA, 25 bp, Homo sapiens
gagcgtttagaacgtttcgcgtttc

SEQ ID NO: 12 - DNA, 27 bp, Homo sapiens
gacgttatggtcgagtgttcgatattc

SEQ ID NO: 13 - DNA, 20 bp, Homo sapiens
gttcgttgggtaaggcgttc

SEQ ID NO: 14 - DNA, 29 bp, Homo sapiens
gtagttatttgagagaacgtcgagtagtc

SEQ ID NO: 15 - DNA, 22 bp, Homo sapiens
gttgtattttcgcggagcgttc

SEQ ID NO: 16 - DNA, 19 bp, Homo sapiens
cgccaaacgaacgaaaccg

SEQ ID NO: 17 - DNA, 20 bp, Homo sapiens
cgaaacctcgccgaaatacg

SEQ ID NO: 18 - DNA, 21 bp, Homo sapiens
atacgcaccattttatcgacc

SEQ ID NO: 19 - DNA, 16 bp, Homo sapiens
gacaacgaccgcgacg

SEQ ID NO: 20 - DNA, 26 bp, Homo sapiens
aacgacctctaaaaaccgaatcaacg

SEQ ID NO: 21 - DNA, 24 bp, Homo sapiens
ctctccgctccaaacgctaacgcg

SEQ ID NO: 22 - DNA, 22 bp, Homo sapiens
cgctaccgatatccgctaaacg

SEQ ID NO: 23 - DNA, 24 bp, Homo sapiens
cgtaaaccccgcccccttatatcg

SEQ ID NO: 24 - DNA, 19 bp, Homo sapiens
accgtcttctcgaacgacg

SEQ ID NO: 25 - DNA, 22 bp, Homo sapiens
accacgtccgaaaaaatcgacg

SEQ ID NO: 26 - DNA, 26 bp, Homo sapiens
aaaatcgctaacgtaaacgttcgacg

SEQ ID NO: 27 - DNA, 27 bp, Homo sapiens
caaacccacaaacgatccgaataatcg

SEQ ID NO: 28 - DNA, 22 bp, Homo sapiens
cataaaacgaacacccgaaccg

SEQ ID NO: 29 - DNA, 24 bp, Homo sapiens
gaacaaaccgactcgaaaacaacg

SEQ ID NO: 30 - DNA, 22 bp, Homo sapiens
ctcacaatacgtctaaccgacg

SEQ ID NO: 31 - DNA, 84 bp, Homo sapiens
gccgtccggggcgagcaccggagcccgactgggctgaatggcaggtcctgaccaagccacccgcgcggccccgcccgcctggcg

SEQ ID NO: 32 - DNA, 89 bp, Homo sapiens
ctagagaccagcctcggtcttcggcctgcgggttctgcaaagtcaggctagctggctctccgcctgctccgcaccccggcgaggttccg

SEQ ID NO: 33 - DNA, 98 bp, Homo sapiens
gcgcggctcatcccagcgccacttgctctgcagctcccagaggtggtggttgtgttacgaaggctgaccctgccaatggccgacaaaatggtgcgcac

SEQ ID NO: 34 - DNA, 117 bp, Homo sapiens
cgggctcgcaacgctgggctcagcgctcgcgcctccctcagctctctcctccgccccccttcgccctccccctttccctccctttctcctcctcctcctgccgccgcggccgctgcc

SEQ ID NO: 35 - DNA, 75 bp, Homo sapiens
cccgccggagagggggccgggccggcgccgctcgctcagagcccagactcgctgacccggctcctagaggccgcc

SEQ ID NO: 36 - DNA, 120 bp, Homo sapiens
gcgacaggcctccagcaagcaagcgcgggcggcatccgcagtctccagaagtttgagacttggccgtaagcggactcgtgcgccccaactctttgccgcgccagcgcctggagcggagag

SEQ ID NO: 37 - DNA, 72 bp, Homo sapiens
gctgtgagctgcgctctccacgccggctccgcgctccaggggctgctgagcgcccagcggacaccggcagcg

SEQ ID NO: 38 - DNA, 98 bp, Homo sapiens
actcgcacgacatggtgacgggcttccgagccttcgaggactagggaaactgtgagcgggaggggctttatacccgacataagggggcggggcccacg

SEQ ID NO: 39 - DNA, 105 bp, Homo sapiens
gtcggctgacgctttgagacaagccggaaaagggccgggttcgccgaaggccgcgtaatccacctggccgctgaggaggaaagagccgccgcccgagaagacggc

SEQ ID NO: 40 - DNA, 102 bp, Homo sapiens
taaagacgcctccgcggccaaggccgaaaggggaagcgaggaggccgccggggtgagtgccctcgggtgtagagagaggacgccgatttccccggacgtggt

SEQ ID NO: 41 - DNA, 150 bp, Homo sapiens
gagcgcccagaacgccccgcgctccgccgagccccgctccacgcagacccgcgggcgggagggagccacgcacatcgccgccgcggccgtctccgcggggcggtaaccgagcctgcctcggagccgccgaacgcccacgccagcgaccct

SEQ ID NO: 42 - DNA, 68 bp, Homo sapiens
gacgccatggccgagtgcccgacactcggggaggcagtcaccgaccacccggaccgcctgtgggcctg

SEQ ID NO: 43 - DNA, 87 bp, Homo sapiens
gcccgctgggcaaggcgtccgagaaagcgcctggcgggaggaggtgcgcggctttctgctccaggcggcccgggtgcccgctttatg

SEQ ID NO: 44 - DNA, 70 bp, Homo sapiens
gcagtcacctgagagaacgccgagcagccgcctggctgcgctttctcgctgcctccgagccggcctgccc

SEQ ID NO: 45 - DNA, 108 bp, Homo sapiens
gctgcatcttcgcggagcgcccggcagaaatagcctagggaagacgaaaaacagctagcggagccgcccaggctgcagctataaagcgccggccagacgcactgtgag

SEQ ID NO: 46 - DNA, 20 bp, Homo sapiens
gttcgttgggtaaggcgttc

SEQ ID NO: 47 - DNA, 22 bp, Homo sapiens
cataaaacgaacacccgaaccg

SEQ ID NO: 48 - DNA, 20 bp, Homo sapiens
gttcgttgggtaaggcgttc

SEQ ID NO: 49 - DNA, 22 bp, Homo sapiens
cataaaacgaacacccgaaccg

SEQ ID NO: 50 - DNA, 62 bp, Homo sapiens
cgacatgcaccgcgcacctcctcccgccaagcatgtcgttagatttcgtaaacggtgaaaac

SEQ ID NO: 51 - DNA, 19 bp, Homo sapiens
tctcctccgaaaaacgctc

SEQ ID NO: 52 - DNA, 33 bp, Homo sapiens
cgtctgcaaccgccgacgaccgcgacgcagacg